(19) United States
(12) Patent Application Publication
(10) Pub. No.: US 2025/0325573 A1
(43) Pub. Date: Oct. 23, 2025

(54) Anti-Cancer Saccharide-Linked Dithiocarbamate Compounds

(71) Applicant: Anglia Ruskin University Higher Education Corporation, Chelmsford, Essex (GB)

(72) Inventor: Mohammad Najlah, Chelmsford, Essex (GB)

(21) Appl. No.: 18/870,360

(22) PCT Filed: May 31, 2023
(86) PCT No.: PCT/EP2023/064609

§ 371 Date: Nov. 27, 2024

(30) Foreign Application Priority Data
May 31, 2022 (GB) 2208040.2

(51) Int. Cl. A61K 31/7028; A61K 33/30; A61K 33/34; A61P 35/00

(52) U.S. Cl. CPC A61K 31/7028; A61K 33/30; A61K 33/34; A61P 35/00

(57) ABSTRACT

The present invention provides a compound, or the pharmaceutically acceptable salts or solvates thereof, for use in the treatment of a subject with cancer, wherein the compound has the general formula (I) wherein each of Rⁿ¹ and Rⁿ² is independently selected from the group consisting of C₁-C₆ alkyl and C₂-C₆ alkenyl; A is a (poly)saccharide connected via a thioglycosidic bond; and x is 1 or more. Also provided is a pharmaceutical composition for use in the treatment of a subject with cancer, the pharmaceutical composition comprising the compound of formula (I), optionally further comprising a metal, wherein the pharmaceutical composition is for simultaneous administration of the compound of formula (I) and the metal.

### RELATED APPLICATION

The present application claims the benefit of and priority to GB 2208040.2, filed on 31 May 2022 (31.05.2022), the contents of which are hereby incorporated by reference in their entirety.

### FIELD OF THE INVENTION

The present invention relates to a compound and a pharmaceutical composition comprising thereof for the treatment of a subject with cancer.

### BACKGROUND

One of the most important medical challenges to date is the treatment of cancer. Despite recent advances and development in the field, cancer remains as one of the dominant causes of death worldwide. According to data provided by the Global Cancer Observatory, there were more than 19.2 million new cases and more than 9.9 million deaths in 2020, with a projected estimation of 30.2 million new cases and 16.3 million deaths in 2040. Such statistics is evident to have a major impact on global communities and is especially significant for low- and middle-income countries, which are least prepared for the challenge.

Disulfiram is known to have anti-cancer properties. Anti-cancer activity arises when disulfiram dissociates from into its metabolite diethyldithiocarbamate in the presence of metal ions, such as copper ions in the body which then form a Cu(II) bis(N,N-diethyldithiocarbamate) complex (DDC)₂Cu. This complex is the active anti-cancer agent which suppresses cancer stem cells by targeting aldehyde dehydrogenase, a marker of cancer stem cells, and inhibits proteasome activity in cancer cells.

There has however previously been little clinical success in using disulfiram as an anti-cancer agent (Kannappan et al. Front Mol Biosci. 2021; 8:7411316). In trials, the inherent insolubility and instability of disulfiram in blood plasma is a drawback of its use, thereby posing challenges in its formulation and administration. Its metabolite, diethyldithiocarbamate, is readily metabolised in the blood, thereby reducing the chelating ability to form the complex. These issues limit the use of disulfiram.

The use of an aqueous core of liposomes to develop an injectable anti-cancer formulation has been described as an attempt to overcome the problems associated with the insolubility of disulfiram (Wehbe et al. Int. J. Nanomedicine. 2017; 12; 4129-4146). However, this method requires formulating disulfiram to protect it from degradation during administration and delivery, and so consequently introduces more involved steps to prepare the prodrug prior to its use in cancer treatment.

Furthermore, the anti-cancer activity may also be improved on. In addition to stability issues surrounding disulfiram, its product of metabolism, S-methyl diethyldithiocarbamate, is stable such that it becomes unreactive as a ligand towards copper ions due to the loss of its chelation ability. This effectively limits the formation of the active complex, thereby limiting its anti-cancer activity.

Dithiocarbamate compounds are also known to have anti-bacterial properties and anti-carcinogenic activity. For example, sugar chain derivatives of 2-acetamido-2-deoxy-β-D-glucopyranosyl N,N-dimethyldithiocarbamates have anti-tuberculous activities against tubercle bacillus (JP 2009-242376 A), and against Mycobacterium tuberculosis (Horita et al. Bioorg. Med. Chem. Lett; 2009; 19 (12); 6313-6316). Dithiocarbamates including diethyldithiocarbamate (DDC), lactose-DDC, proline-dithiocarbamate and 4-carboxy-piperazine-TDS are shown to be promising agents in the chemoprevention of liver carcinogenesis caused by aflatoxin B1 (Gopalaswamy et al. Anticancer Res. 1998; 18 (3A); 1827-1832).

Accordingly, there is a need for a new method of treating cancer using a compound which can exhibit excellent anti-cancer activity while having excellent in vivo and in vitro solubility and stability.

The present invention has been devised in the light of the above considerations.

### SUMMARY OF THE INVENTION

The present inventors have found that saccharide-linked dithiocarbamates may act as a prodrug which breaks down in vivo to form an active complex, and these dithiocarbamates may be used in the treatment of a subject who has cancer. The compounds of the invention may be regarded as having one or more thiocarbamate groups connected to a (poly)saccharide connected to each thiocarbamate via a thioglycosidic bond.

In particular, the saccharide protects the dithiocarbamate from metabolism and provides a prodrug that is significantly more stable and soluble in the blood stream than disulfiram, while still maintaining the ability to be cleaved by metal ions and the chelating ability dithiocarbamate ligand to form the active copper complex to thereby exhibit excellent anti-cancer activity.

Accordingly, in a first aspect of the present invention, there is provided a compound, or the pharmaceutically acceptable salts or solvates thereof, for use in the treatment of a subject having cancer, wherein the compound has the general formula (I):

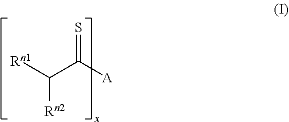

wherein each of Rⁿ¹ and Rⁿ² is independently selected from the group consisting of C₁-C₆ alkyl and C₂-C₆ alkenyl;
A is a (poly)saccharide connected via a thioglycosidic bond; and
x is 1 or more, such as x is 1 or x is 2, 3, 4, 5, 6, 7, 8, 9 or 10.

Such a compound may be referred to as a dithiocarbamate by virtue of the connection of the thiocarbamate group in formula (I) to the (poly)saccharide via a thioether bond.

The present inventors have established that the compound of general formula (I) is advantageous in providing excellent anti-cancer activity to a wide range of cancer cell lines, including resistant cancer cell lines such as colorectal cancer cell line H360 R10, and improved solubility and stability in aqueous media.

The groups Rⁿ¹ and Rⁿ² may be selected from ethyl and methyl, such as each of Rⁿ¹ and Rⁿ² is ethyl or each of Rⁿ¹ and Rⁿ² is methyl.

Preferably, x is 1 or 2, such as x is 1.

Preferably, A is a monosaccharide connected via a thioglycosidic bond.

More preferably, A is:

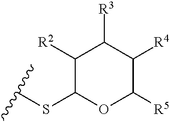

wherein R² to R⁴ is each —OH, or wherein R² to R⁴ is each —OH and at least one —OH, such as one, is replaced with a group independently selected from H, —NH₂, acetoxy (—OAc), acetylamido (—NHAc), —OBn and —OBz; and
R⁵ is H, or —CH₂OH, or wherein R⁵ is —CH₂OH and —OH is replaced with a group independently selected from H (thus, R⁵ is —CH₃), —NH₂ (thus, R⁵ is —CH₂NH₂), acetoxy (—OAc), acetylamido (—NHAc), —OBn and —OBz.

In an alternative embodiment, A is a disaccharide, an oligosaccharide or a polysaccharide connected via a thioglycosidic bond.

In a second aspect of the present invention, there is provided a pharmaceutical composition for use in the treatment of a subject having cancer, the pharmaceutical composition comprising a compound of general formula (I) and a pharmaceutically acceptable carrier.

The present inventors have found that a pharmaceutically acceptable carrier for the compound is beneficial for providing improved permeability through the cellular membrane to a target cancer cell.

The pharmaceutical composition for use may also comprise a metal, such as a metal ion, selected from the group consisting of copper, zinc, platinum, iron, gold, silver and magnesium, and preferably copper.

This allows for simultaneous administration of the pharmaceutical composition and the metal ion as a supplement to a subject with cancer.

The pharmaceutical composition for use may be used in the treatment of a cancer selected from the group consisting of colorectal cancer, breast cancer, lung cancer and brain cancer.

The invention also provides a kit comprising a pharmaceutical composition comprising the compound for use and a pharmaceutically acceptable carrier or excipient, and a metal, such as metal ion, selected from the group consisting of copper, zinc, platinum, iron, gold, silver and magnesium, and preferably copper.

This allows for separate administration of the pharmaceutical composition and metal as a supplement to a subject with cancer.

In a third aspect of the present invention, there is provided a method of forming a complex, such as ex vivo, the method comprising contacting the compound of general formula (I) with a metal, such as a metal ion, selected from the group consisting of copper, zinc, platinum, iron, gold, silver and magnesium, and the ions thereof, and preferably copper ion, to give a complex of the metal with dithiocarbamate, and a (poly)saccharide cleavage product.

The complex formed from the method may the active agent for an anti-cancer treatment. The present inventors have found that the complex is formed rapidly from the compound of formula (I), which may be regarded as a prodrug, and a metal in an aqueous environment, such as an intracellular environment, such as the intracellular environment of a cancer cell. It has also believed that the (poly)saccharide cleavage product provides an additional anti-cancer effect.

It will be understood that the compound of general formula (I) and a metal supplement may be administered together or separately and may be administered at the same time, or at different times. For example, the compound of general formula (I) may be administered, and metal may be administered only if the subject with cancer requires a supplement of metal.

The choice of administration may be dependent on the requirements of the particular subject.

In a further aspect there is provided a compound of general formula (I) and the pharmaceutically acceptable salts or solvates thereof.

These and other aspects and embodiments of the invention are described in further detail below.

### SUMMARY OF THE FIGURES

Embodiments and experiments illustrating the principles of the invention will now be discussed with reference to the accompanying figures in which:

FIG. 1 shows the stability of glycosyl diethyldithiocarbamate (G-DDC), deoxy-glycosyl diethyldithiocarbamate (DG-DDC), and 2-N-acetyl-glycosylamine diethyldithiocarbamate (AG-DDC) compared to disulfiram (DS) in foetal horse serum as measured by remaining % over time (min).

FIG. 2 shows the survival rates (MTT cytotoxicity assay) of colorectal cancer cell lines H630 WT with increasing concentrations of glycosyl diethyldithiocarbamate (G-DDC) with copper(II) (10 μM) and glycosyl diethyldithiocarbamate (G-DDC) without copper(II).

FIG. 3 shows the survival rates (MTT cytotoxicity assay) of breast cancer cell line MDA-MB-231 with increasing concentrations of glycosyl diethyldithiocarbamate (G-DDC) with copper(II) (10 μM), and glycosyl diethyldithiocarbamate (G-DDC) without copper(II).

FIG. 4 shows the survival rates (MTT cytotoxicity assay) of colorectal cancer cell lines H630 WT and H630 R10 (resistant to 5FU 10 μM) with increasing concentrations of (A) 2-deoxy-glycosyl diethyldithiocarbamate (DG-DDC) with copper(II) (10 μM), and (B) 2-deoxy-glycosyl diethyldithiocarbamate (DG-DDC) without copper(II), and (C) colorectal cancer cell line H630 R10 with increasing concentrations of combination of 2-deoxy-glycosyl diethyldithiocarbamate (DG-DDC) with copper(II) (10 μM) and fluorouracil (5FU).

FIG. 5 shows the survival rates (MTT cytotoxicity assay) of breast cancer cell line MDA-MB-231 with increasing concentrations of 2-deoxy-glycosyl diethyldithiocarbamate (DG-DDC) and 2-deoxy-glucose with copper(II) (10 μM), and 2-deoxy-glycosyl diethyldithiocarbamate (DG-DDC) and 2-deoxy-glucose without copper(II).

FIG. 6 shows the survival rates (MTT cytotoxicity assay) of lung cancer cell line A549 with increasing concentrations of glycosyl diethyldithiocarbamate (G-DDC) with copper(II) (10 μM), 2-deoxy-glycosyl diethyldithiocarbamate (DG-DDC) with copper(II), and 2-deoxy glucose (DG) with copper(II) (10 μM).

FIG. 7 shows the survival rates (MTT cytotoxicity assay) of lung cancer cell line A549 with increasing concentrations of 2-deoxy-glycosyl diethyldithiocarbamate (DG-DDC) with copper(II) (10 μM) and a liposomal formulation of 2-deoxy-glycosyl diethyldithiocarbamate (Lipo DG-DDC) with copper(II) (10 μM).

FIG. 8 shows the survival rates (MTT cytotoxicity assay) of lung cancer cell line A549 with increasing concentrations of 2-deoxy-glycosyl diethyldithiocarbamate (DG-DDC) with zinc (II) (10 μM).

FIG. 9 shows the survival rates (MTT cytotoxicity assay) of colorectal cancer cell line H630 WT with increasing concentrations of 2-N-acetyl-glycosylamine diethyldithiocarbamate (AG-DDC) with copper(II) (10 μM).

FIG. 10 shows the survival rates (MTT cytotoxicity assay) of colorectal cancer cell line H630 WT with increasing concentrations of xylosyl diethyldithiocarbamate (XY-DDC) with copper(II) (10 μM).

FIG. 11 shows the survival rates (MTT cytotoxicity assay) of breast cancer cell line MDA-MB-231 with increasing concentrations of lactosyl diethyldithiocarbamate (La-DDC) with copper(II) (10 μM).

FIG. 12 shows the survival rates (MTT cytotoxicity assay) of breast cancer cell line MDA-MB-231 with increasing concentrations of galactosyl diethyldithiocarbamate (Ga-DDC) with copper(II) (10 μM).

FIG. 13 shows the survival rates (MTT cytotoxicity assay) of breast cancer cell line MDA-MB-231 with increasing concentrations of mannosyl diethyldithiocarbamate (Ma-DDC) with copper(II) (10 μM).

FIG. 14 shows the survival rates (MTT cytotoxicity assay) of breast cancer cell line MDA-MB-231 with increasing concentrations of β-D-glucopyranose, 1-thio-, 2,3,4,6-tetraacetate 1-(N,N-diethylcarbamodithioate) (Tetra-DDC) with copper(II) (10 μM).

FIG. 15 shows the reaction progress of glycosyl diethyldithiocarbamate (G-DDC), disulfiram (DS) in 50:50 DMSO/water, disulfiram (DS) in water, glycosyl diethyldithiocarbamate (DG-DDC), and 2-N-acetylglycosylamine diethyldithicarbamate (AG-DDC) with CuCl₂ to produce the active Cu(II) bis(N,N-diethyldithiocarbamate) complex.

FIG. 16 shows microscopy images of lung cancer cell line A549 at 200× magnification in 0 mM, 2 mM, 4 mM, 8 mM concentrations of glycosyl diethyldithiocarbamate (G-DDC), 2-deoxy-glycosyl diethyldithiocarbamate (DG-DDC) and 2-deoxy-glucose (DG), all without copper(II) after 24 hours.

FIG. 17 shows microscopy images of breast cancer cell line MDA-MB-231 at 100× magnification in glycosyl diethyldithiocarbamate (G-DDC), 2-deoxy-glycosyl diethyldithiocarbamate (DG-DDC) and 2-deoxy-glucose (DG) after 24 hours, all without copper(II).

FIG. 18 shows microscopy images of breast cancer cell line MDA-MB-231 at 100× magnification in 250 μM glycosyl diethyldithiocarbamate (G-DDC), 225 μM 2-deoxy-glycosyl diethyldithiocarbamate (DG-DDC) and 1,000 μM 2-deoxy-glucose (DG) after 24 hours, all with 10 μM copper(II).

FIG. 19 shows microscopy images of lung cancer cell line A549 at 100× magnification in 1 mM glycosyl diethyldithiocarbamate (G-DDC), 1 mM 2-deoxy-glycosyl diethyldithiocarbamate (DG-DDC) and 1 mM 2-deoxy-glucose (DG) after 72 hours, without copper(II), and 0.25 mM glycosyl diethyldithiocarbamate (G-DDC), 0.025 mM 2-deoxy-glycosyl diethyldithiocarbamate (DG-DDC) and 1 mM 2-deoxy-glucose (DG) after 72 hours, with copper(II).

FIG. 20 shows microscopy images of colorectal cancer cell line H630 WT at 100× magnification in glycosyl diethyldithiocarbamate (G-DDC), 2-deoxy-glycosyl diethyldithiocarbamate (DG-DDC) and sodium diethyldithiocarbamate (DDC-Na) after 72 hours, all with 10 μM copper(II).

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### (Poly)saccharides

(Poly)saccharides may include sugars, starch and cellulose and may be found in abundance in a variety of natural and processed foods. Monosaccharides may be linked together via glycosidic linkages to form disaccharides, trisaccharides, oligosaccharides and other higher order polysaccharides.

It is an object of the present invention to improve the stability and solubility of a dithiocarbamate compound for use in the treatment of a subject with cancer. The stability and solubility may be improved by the connection of the dithiocarbamate to a (poly)saccharide via a thioglycosidic bond, as represented by the compound of formula (I). The (poly)saccharide-linked dithiocarbamate is cleavable, releasing the dithiocarbamate and this may form an active complex in the presence of metals, such as metal ions.

Upon dissociation of the compound into a dithiocarbamate and a (poly)saccharide, the regenerated saccharide, described herein as a saccharide cleavage product, may also provide additional anti-cancer effects.

The present inventors have found that a modification to dithiocarbamates by S-linked glycosylation, herein also referred to as a thioglycosidic bond, with a saccharide to obtain the compound may protect the dithiocarbamate from metabolism in the blood while maintaining the cleavage susceptibility of the compound by a metal. The inherent water solubility of saccharides as a result of having multiple hydroxyl groups increases the overall water solubility of the compound, thereby avoiding the requirement for further advanced formulations for drug delivery.

However, an increase in solubility alone is insufficient in providing an improvement in anti-cancer activity. The saccharides are specifically selected because its reaction with a dithiocarbamate introduces a direct substitution of a hydroxyl group at the anomeric position of the saccharide. Without wishing to be bound by theory, it is believed that S-linked glycosylation between the dithiocarbamate and the anomeric position of the saccharide lowers the energy barrier for cleavage of the compound in the presence of a metal, such as copper.

As a result of hyperconjugation arising from the anomeric effect, a cyclohexyl system with the general formula C—X—C—Y, wherein X is an atom having one or more lone pair of electrons, in this case O, and Y is an electronegative atom, in this case S, causes a proportion of the compound having the dithiocarbamate in the axial orientation instead of the less sterically-hindered equatorial position. There is a net stabilisation by donation of a lone of pair of electrons from O to the C—S o⁻* antibonding orbital. In addition to the natural anomerisation between saccharides of cyclic and linear open-chain forms, the net stabilisation from the orbital overlap causes the C—S bond to be more labile in the presence of a metal. This lowers the energy barrier for the cleavage of the compound at the C—S bond to produce the active complex upon coordination of the dithiocarbamate ligand to the metal.

Therefore, the saccharide of the present invention may be a saccharide that is capable of providing improved water solubility compared to disulfiram, allowing the cleavage of the dithiocarbamate from the saccharide in the presence of copper ions and maintaining the chelation ability of the resulting dithiocarbamate ligand upon dissociating from the compound.

The saccharide of the present invention is a saccharide which is connected to the dithiocarbamate by S-linked glycosylation. Accordingly, the saccharide is a thiosaccharide. As described herein, a thiosaccharide is a saccharide comprising a sulfur atom at the anomeric position upon substitution of a hydroxyl group at the anomeric position, wherein the sulfur atom originates from the dithiocarbamate. The thiosaccharide may be a monothiosaccharide, a dithiosaccharide, an oligothiosaccharide or a polythiosaccharide.

Examples of thiosaccharides include monothiosaccharides selected from the group consisting of 1-thio-glucose, 1-thio-mannose, 1-thio-galactose, 1-thio-galactosylamine, 1-thio-xylose, 2-deoxy-1-thio-glucose, 2-O-propyl-1-thio-glucose, 2-N-acetyl-1-thio-glucosamine and 2-N-acetyl-1-thio-galactosamine, dithiosaccharides selected from the group consisting of 1-thio-lactose, 1-thio-maltose and 1-thio-chitobiose, oligothiosaccharides selected from the group consisting of 1-thio-maltotriose and 1-thio-maltopentose, and polythiosaccharides such as 1-thio-chitosan.

The compound has the general formula (I).

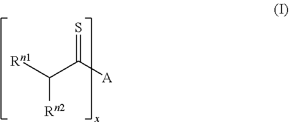

wherein each of Rⁿ¹ and Rⁿ² is independently selected from the group consisting of C₁-C₆ alkyl and C₂-C₆ alkenyl; A is a (poly)saccharide connected via a thioglycosidic bond; and x is 1 or more, such as x is 1 or x is 2, 3, 4, 5, 6, 7, 8, 9 or 10. The term “(poly)saccharide” should be understood to include monosaccharides, disaccharides, oligosaccharides and polysaccharides.

Preferably, A is a monosaccharide connected via a thioglycosidic bond. More preferably, A is a monosaccharide which is a hexose or pentose connected via a thioglycosidic bond, herein also referred to as a 1-thiohexose or a 1-thiopentose, respectively. A hexose is a monosaccharide with six carbon atoms. A pentose is a monosaccharide with five carbons. Preferably, both the hexose and the pentose are in the pyranose form.

More preferably, A is:

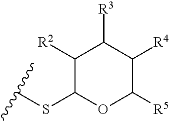

wherein R² to R⁴ is each —OH, or wherein R² to R⁴ is each —OH and at least one —OH, such as one, is replaced with a group independently selected from H, —NH₂, acetoxy, acetylamido, —OBn and —OBz; and
R⁵ is H, or —CH₂OH, or wherein R⁵ is —CH₂OH and —OH is replaced with a group independently selected from H, —NH₂, acetoxy, acetylamido, —OBn and —OBz.

In one embodiment, each of R² to R⁴ is —OH, and R⁵ is —CH₂OH, such that A is 1-thio-glycosyl, 1-thio-mannosyl or 1-thio-galactosyl.

In another embodiment, R² is H, each of R³ and R⁴ is —OH, and R⁵ is —CH₂OH, such that A is 2-deoxy-1-thio-glycosyl.

In a further embodiment, R² is acetylamido, each of R³ and R⁴ is —OH, and R⁵ is —CH₂OH, such that A is 2-N-acetyl-1-thio-glycosylamine or 2-N-acetyl-1-thio-galactosylamine.

In one embodiment, R² is —NH₂ each of R³ and R⁴ is —OH, and R⁵ is —CH₂OH, such that A is 1-thio-galactosylamine.

In one embodiment, R² is O-propyl (—OPr), each of R³ and R⁴ is —OH, and R⁵ is —CH₂OH, such that A is 2-O-propyl-1-thio-glycosyl. The propyl group may be n-propyl or i-propyl.

In another embodiment, R² is H, R³ is acetoxy, R⁴ is —OH, and R⁵ is acetoxy, such that A is 1-thio-3,6-diacetate-2-deoxy-glycosyl.

The groups R² to R⁴ may be —OH, and at least one —OH group is replaced with a group independently selected from H, amino, acetoxy, acetylamido, —OBn and —OBz. Here, R⁵ is —CH₂OH, or R⁵ may be —CH₂OH where —OH is replaced with a group independently selected from H, amino, acetoxy, acetylamido, —OBn and —OBz.

The groups R² to R⁴ may be —OH, and two-OH groups are replaced with a group independently selected from H, amino, acetoxy, acetylamido, —OBn and —OBz. Here, R⁵ is —CH₂OH, or R⁵ may be —CH₂OH where —OH is replaced with a group independently selected from H, amino, acetoxy, acetylamido, —OBn and —OBz.

Each of R² to R⁴ is —OH where every —OH group is replaced with a group independently selected from H, amino, acetoxy, acetylamido, —OBn and —OBz. Here, R⁵ is —CH₂OH, or R⁵ may be —CH₂OH where —OH is replaced with a group independently selected from H, amino, acetoxy, acetylamido, —OBn and —OBz.

In one embodiment, R⁵ is —CH₂OH, and each of R² to R⁴ is —OH, where the —PH group is replaced with a group independently selected from H, amino, acetoxy, acetylamido, —OBn and —OBz.

In one embodiment, R³ and R⁴ is each —OH, R⁵ is —CH₂OH, and R² is selected from the group consisting of H, —NH₂, acetoxy, acetylamido, —OBn and —OBz (these groups formally replacing-OH at this position).

The group A may be a monosaccharide which is a pentose connected via a thioglycosidic bond, (a 1-thiopentose). A pentose is a monosaccharide with five carbon atoms. A pentose may be a monosaccharide having the general formula shown above, where the group R⁵ is H.

When A is a pentose connected via a thioglycosidic bond, each of R² to R⁴ may be —OH, or wherein R² to R⁴ is each —OH and at least one —OH, such as one, is replaced with a group independently selected from H, —NH₂, acetoxy, acetylamido, —OBn and —OBz. The group R⁵ is H. For example, R² to R⁴ is each —OH, and R⁵ is H, such that A is 1-thio-xylosyl.

A compound for use in the invention may be one of the compounds selected from Table 1A and 1B, such as those selected from Table 2A and 2B, and the pharmaceutically acceptable salts or solvates thereof.

TABLE 1A
Summary of preferred compounds.

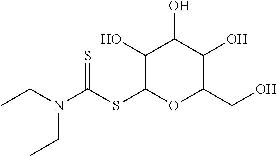 [SMILES: CCN(CC)C(=S)SC1OC(CO)C(O)C(O)C1O]

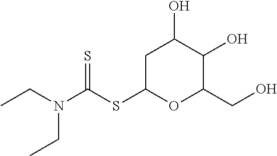 [SMILES: CCN(CC)C(=S)SC1CC(O)C(O)C(CO)O1]

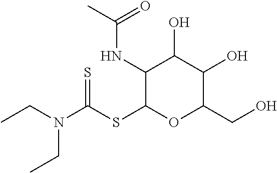 [SMILES: CCN(CC)C(=S)SC1OC(CO)C(O)C(O)C1NC(C)=O]

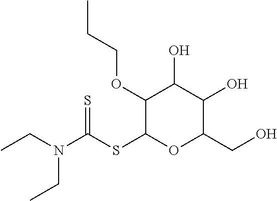 [SMILES: CCCOC1C(SC(=S)N(CC)CC)OC(CO)C(O)C1O]

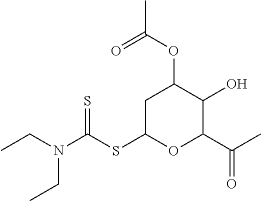 [SMILES: CCN(CC)C(=S)SC1CC(OC(C)=O)C(O)C(C(C)=O)O1]

TABLE 1B
Summary of additional preferred compounds.

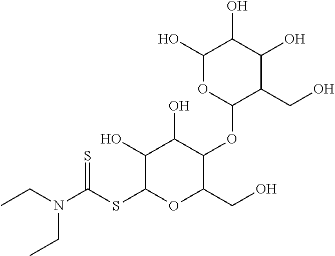 [SMILES: CCN(CC)C(=S)SC1OC(CO)C(OC2OC(O)C(O)C(O)C2CO)C(O)C1O]

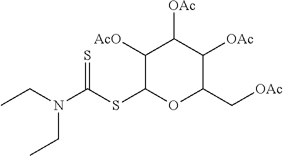 [SMILES: CCN(CC)C(=S)SC1OC(COC(C)=O)C(OC(C)=O)C(OC(C)=O)C1OC(C)=O]

TABLE 2A
Summary of preferred compounds showing
bond stereochemistry.

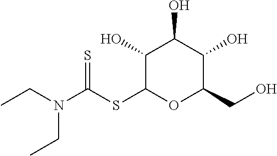 [SMILES: CCN(CC)C(=S)SC1O[C@H](CO)[C@@H](O)[C@H](O)[C@H]1O]

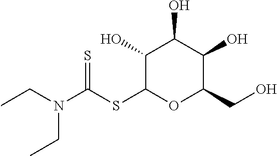 [SMILES: CCN(CC)C(=S)SC1O[C@H](CO)[C@H](O)[C@H](O)[C@H]1O]

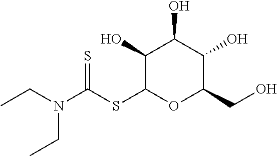 [SMILES: CCN(CC)C(=S)SC1O[C@H](CO)[C@@H](O)[C@H](O)[C@@H]1O]

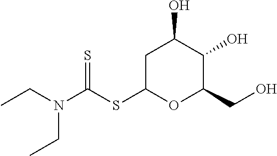 [SMILES: CCN(CC)C(=S)SC1C[C@@H](O)[C@H](O)[C@@H](CO)O1]

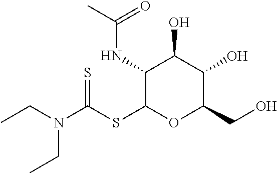 [SMILES: CCN(CC)C(=S)SC1O[C@H](CO)[C@@H](O)[C@H](O)[C@H]1NC(C)=O]

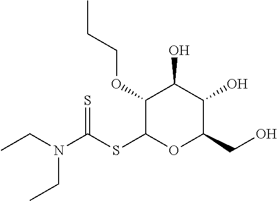 [SMILES: CCCO[C@H]1C(SC(=S)N(CC)CC)O[C@H](CO)[C@@H](O)[C@@H]1O]

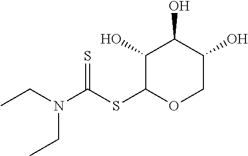 [SMILES: CCN(CC)C(=S)SC1OC[C@@H](O)[C@H](O)[C@H]1O]

TABLE 2B
Summary of additional preferred compounds
showing bond stereochemistry.

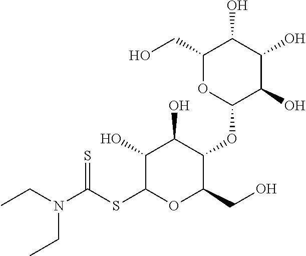 [SMILES: CCN(CC)C(=S)SC1O[C@H](CO)[C@@H](O[C@@H]2O[C@H](CO)[C@H](O)[C@H](O)[C@H]2O)[C@H](O)[C@H]1O]

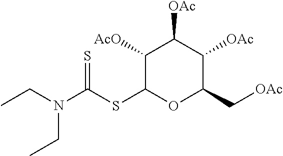 [SMILES: CCN(CC)C(=S)SC1O[C@H](COC(C)=O)[C@@H](OC(C)=O)[C@H](OC(C)=O)[C@H]1OC(C)=O]

It is particularly preferable that A is selected from the group consisting of 1-thio-glycosyl, 1-thio-mannosyl, 1-thio-galactosyl, 2-deoxy-1-thio-glycosyl, 2-N-acetyl-1-thio-glycosylamine, 2-N-acetyl-1-thio-galactosylamine, 2-O-propyl-1-thio-glycosyl and 1-thio-3,6-diacetate-2-deoxy-glycosyl.

Alternatively, A is a disaccharide, oligosaccharide or polysaccharide connected via a thioglycosidic bond. Preferably, A is selected from the group consisting of 1-thio-lactosyl, 1-thio-maltosyl, 1-thio-chitobiosyl, 1-thio-maltotriosyl, 1-thio-maltopentosyl and 1-thiochitosanyl. In such embodiments, A is a (poly)saccharide selected from the list consisting of 4-O-galactopyranosyl-1-thio-glycosyl, 4-O-glucopyranosyl-1-thio-glycosyl, 4-O-(2-amino-2-deoxy-glucopyranosyl)-2-amino-2-deoxy-1-thio-glycosyl, 4-O-maltosyl-1-thio-glycosyl, 4-O-maltosyl-4-O-maltosyl-1-thio-glycosyl and 1-thiochitosanyl connected via a thioglycosidic bond at a terminal ring.

Accordingly, A may be a saccharide connected via a thioglycosidic bond which is a thiosaccharide selected from the group consisting of 1-thio-glycosyl, 1-thio-mannosyl, 1-thio-galactosyl, 1-thio-galactosylamine, 1-thio-xylosyl, 2-deoxy-1-thio-glycosyl, 2-O-propyl-1-thio-glycosyl, 2-N-acetyl-1-thio-glycosylamine, 2-N-acetyl-1-thio-galactosylamine, 1-thio-lactosyl, 1-thio-maltosyl, 1-thio-chitobiosyl, 1-thio-maltotriosyl, 1-thio-maltopentosyl, and 1-thio-chitosanyl at a terminal ring.

It is thought that a compound comprising two dithiocarbamate moieties may further increase anti-cancer activity compared to a compound comprising one dithiocarbamate moiety. Thus, the value of x may be more than 1, and is preferably, x is 2.

The thiosaccharide may exist in the form of two anomers, a and B, which may interconvert, such as which interconvert, via an anomerisation mechanism. As described herein, “1-thiol” with respect to the compound of general formula (I) may also refer to the thioglycosidic bond on the saccharide. The α-anomer of the thiosaccharide comprises a 1-thiol in the axial position of the ring and the β-anomer of the thiosaccharide comprises a 1-thiol in the equatorial position of the ring.

In some embodiments, the thiosaccaride is an α-anomer.

In alternative embodiments, the thiosaccaride is a β-anomer.

Preferably, the thiosaccharide is a monothiosaccharide selected from the group consisting of 1-thio-α-glucose, 1-thio-β-glucose, 1-thio-α-mannose, 1-thio-β-mannose, 1-thio-α-mannose, 1-thio-β-galactose, 1-thio-α-galactosamine, 1-thio-β-galactosamine, 1-thio-α-xylose, 1-thio-β-xylose, 2-deoxy-1-thio-α-glucose, 2-deoxy-1-thio-β-glucose, 2-O-propyl-1-thio-α-glucose, 2-O-propyl-1-thio-β-glucose, 2-N-acetyl-1-thio-α-glucosamine, 2-N-acetyl-1-thio-β-glucosamine, 2-N-acetyl-1-thio-α-galactosamine and 2-N-acetyl-1-thio-β-galactosylamine, a dithiosaccharide selected from the group consisting of 1-thio-α-lactose, 1-thio-β-lactose, 1-thio-α-maltose, 1-thio-β-maltose, thio-α-chitobiose and 1-thio-β-chitobiose, an oligothiosaccharide selected from the group consisting of 1-thio-α-maltotriose, 1-thio-β-maltotriose, 1-thio-α-maltopentose and 1-thio-β-maltopentose, or a polythiosaccharide such as 1-thio-α-chitosan or 1-thio-β-chitosan.

More preferably, the thiosaccharide is a monothiosaccharide selected from the group consisting of 1-thio-α-glucose, 1-thio-α-mannose, 1-thio-α-galactose, 1-thio-α-galactosamine, 1-thio-α-xylose, 2-deoxy-1-thio-α-glucose, 2-O-propyl-1-thio-α-glucose, 2-N-acetyl-1-thio-α-glucosamine, 2-N-acetyl-1-thio-α-galactosamine, a dithiosaccharide selected from the group consisting of 1-thio-α-lactose, 1-thio-α-maltose, 1-thio-α-chitobiose, an oligothiosaccharide selected from the group consisting of 1-thio-α-maltotriose, 1-thio-α-maltopentose, or a polythiosaccharide such as 1-thio-α-chitosan.

More preferably, the saccharide is a monothiosaccharide selected from the group consisting of 1-thio-α-glucose, 1-thio-α-mannose, 1-thio-α-galactose, 1-thio-α-galactosamine, 1-thio-α-xylose, 2-deoxy-1-thio-α-glucose, 2-O-propyl-1-thio-α-glucose, 2-N-acetyl-1-thio-α-glucosamine and 2-N-acetyl-1-thio-α-galactosamine.

Saccharides and thiosaccharides exhibit chirality such that they may occur as D isomers or L isomers. It should be understood by the skilled person that most naturally occurring saccharides are D isomers. Preferably, from the point of view of the ease of extraction and acquisition from natural products, and so to avoid the need for any particularly complicated laboratory synthesis, the thiosaccharide is present in the D-isomeric form. The thiosaccharide may also be present in the L-isomeric form, although there may be no particular benefit associated with it alone.

Therefore preferably, the saccharide is a monothiosaccharide selected from the group consisting of 1-thio-β-D-glucose, 1-thio-β-D-mannose, 1-thio-β-D-galactose, 1-thio-β-D-galactosamine, 1-thio-β-D-xylose, 2-deoxy-1-thio-3-D-glucose, 2-O-propyl-1-thio-β-D-glucose, 2-N-acetyl-1-thio-β-D-glucosamine and 2-N-acetyl-1-thio-β-D-galactosamine.

More preferably, the saccharide is a monothiosaccharide selected from the group consisting 1-thio-α-D-glucose, 1-thio-α-D-mannose, 1-thio-α-D-galactose, 1-thio-α-D-galactosamine, 1-thio-α-D-xylose, 2-deoxy-1-thio-α-D-glucose, 2-O-propyl-1-thio-α-D-glucose, 2-N-acetyl-1-thio-α-D-glucosamine and 2-N-acetyl-1-thio-α-D-galactosamine.

Pyranose forms of thiosaccharides comprise a six-membered ring consisting of five carbon atoms and one oxygen atom. Each of 1-thio-D-glucose, 1-thio-D-mannose, 1-thio-D-galactose, 1-thio-D-galactosamine, 1-thio-D-xylose, 2-deoxy-1-thio-D-glucose, 2-O-propyl-1-thio-D-glucose, 2-N-acetyl-1-thio-D-glucosamine and 2-N-acetyl-1-thio-D-galactosamine comprises, where present, a ring in the pyranose form.

Preferably, for dithiosaccharides, oligothiosaccharides and polythiosaccharides which comprise more than one ring, each of 1-thio-lactose, 1-thio-maltose, 1-thio-chitobiose, 1-thio-maltotriose, 1-thio-maltopentose, and 1-thio-chitosan comprises, where present, all its rings in the pyranose form.

In view of an object of the present invention, particularly preferred thiosaccharides include 2-deoxy-1-thio-α-D-glucose, which is a monothiosaccharide, and 1-thio-α-chitosan, which is a polythiosaccharide. The respective saccharide cleavage products, 2-deoxy-D-glucose and chitosan, are known to have anti-cancer properties (Aft et al. Br J Cancer. 2002; 87; 805-812 and Shakil et al. Polysaccharides. 2021; 2; 197-816). Advantageously, the combination of a copper complex and a 2-deoxy-D-glucose saccharide cleavage product or a chitosan saccharide cleavage product, both of which are generated upon providing the compound in the presence of copper, may provide a further anti-cancer effect in addition to that of the active complex. In this preferred embodiment, the compound may dissociate to effectively form two active compounds or ingredients having anti-cancer properties.

In some embodiments, each —OH of the thiosaccharide is replaced with acetoxy (—OAc), —OBn or —OBz, or other suitable alcohol protecting groups. In some embodiments, each —OH of the thiosaccharide is replaced with —OAc. In such embodiments, the compound is β-D-glucopyranose, 1-thio-,2,3,4,6-tetraacetate 1-(N,N-diethylcarbamodithioate).

The synthesis of saccharide-linked dithiocarbamates have been previously synthesised and characterised before in the art (Li et al. Tetrahedron Letters. 2016; 57 (31) 3529-3531). The synthesis involves the single step of a direct introduction of a dithiocarbamate group into the anomeric centre of a sugar.

Accordingly, in the present invention, the saccharide is covalently attached via S-linked glycosylation to the dithiocarbamate. The S-linked glycosylation is the connection between an anomeric carbon atom of the saccharide with a dithiocarbamate sulfur atom of the dithiocarbamate to thereby form the thiosaccharide moiety as depicted by A. This involves the substitution of a hydroxyl group on the anomeric carbon atom of the saccharide with the dithiocarbamate functional group of the dithiocarbamate.

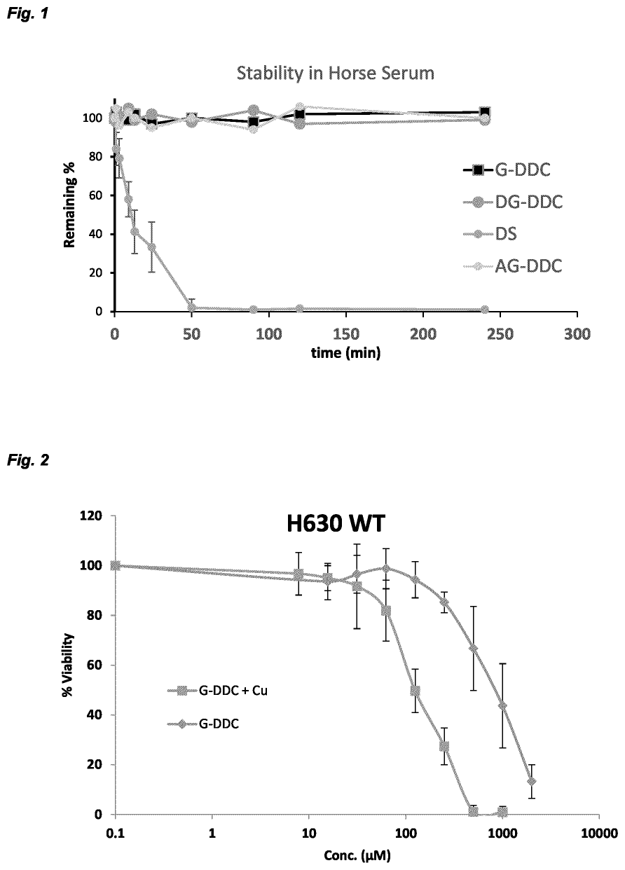

The compound is shown to have improved stability. For example, the stability of glycosyl diethyldithiocarbamates is demonstrated, in FIG. 1, to be significantly improved when compared with the stability of disulfiram in foetal horse serum. Advantageously, the increased stability allows for direct administration, for example to a subject with cancer, without the need for any advanced formulations, unlike the present case for the use of disulfiram as an anti-cancer drug.

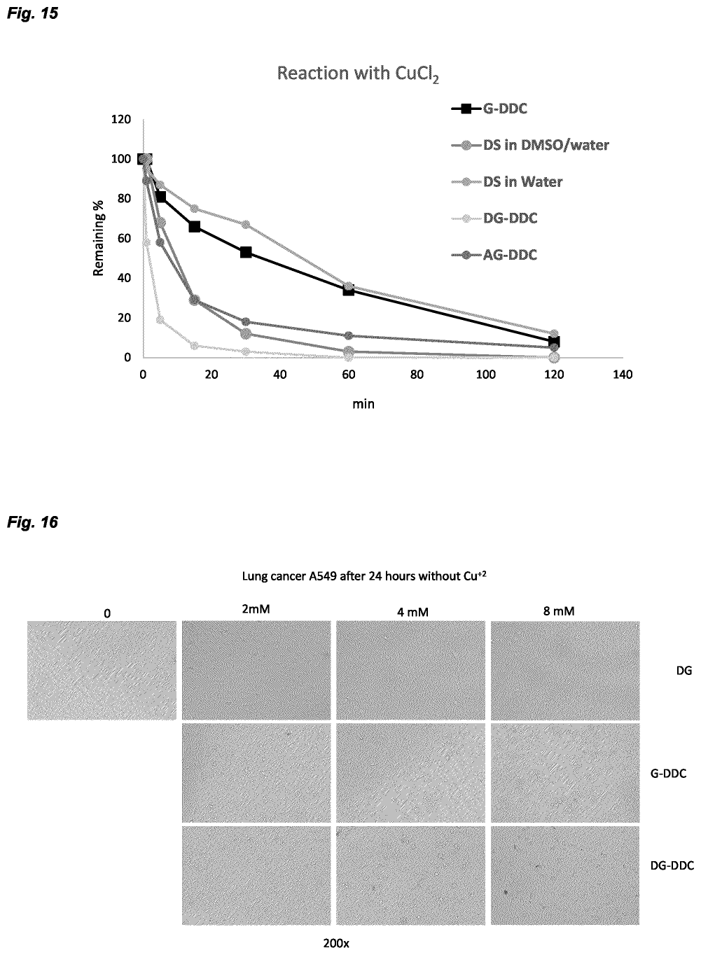

FIG. 15 shows that the reaction between glycosyl diethyldithiocarbamate, 2-deoxy-glycosyl diethyldithiocarbamate and 2-N-acetyl-glycosylamine diethyldithiocarbamate with copper(II) chloride is fast relative to the reaction between disulfiram and copper(II) chloride, further indicating the increased solubility of the compound in water. Advantageously, the increased solubility allows for improved ease of administration.

Accordingly, the combination of the saccharide and dithiocarbamate produces the compound, which is the prodrug. The term “prodrug”, as used herein, pertains to a compound which, when metabolised (e.g., in vivo) yields the desired active compound or ingredient. Typically, the prodrug is inactive, or less active than the active compound or ingredient, but may provide advantageous handling, administration, or metabolic properties. Accordingly, the active compound or ingredient in the method of treatment of the present invention is the Cu(II) bis(N,N-diethyldithiocarbamate) complex or Zn(II) bis(N,N-diethyldithiocarbamate) complex.

Unless otherwise specified, a reference to a particular compound also includes prodrugs thereof.

### Dithiocarbamates

The compounds of the present invention have the general formula (I):

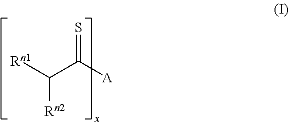

wherein each of Rⁿ¹ and Rⁿ² is independently selected from the group consisting of C₁-C₆ alkyl and C₂-C₆ alkenyl;
A is a (poly)saccharide connected via a thioglycosidic bond, and
x is 1 or more, such as x is 1 or x is 2, 3, 4, 5, 6, 7, 8, 9, or 10.

The present prevention provides compounds of formula (I) as such, and their use in the treatment of a subject with cancer. Also provides are compositions and kits comprising the compounds of formula (I)

The term “x” denotes the number of thiocarbamate moieties present in the compound, each connected to the (poly)saccharide via a thioglycosidic bond. As each connection to the (poly)saccharide is through a thio-bond, each thiocarbamate group may be regarded as a dithiocarbamate, and it is referred to as such below.

The term “C₁-C₆ alkyl” refers to a linear or branched alkyl group with 1 to 6 carbon atoms, such as, but not limited to, methyl, ethyl, propyl, isopropyl, butyl, isobutyl, sec-butyl, tert-butyl, n-amyl, iso-amyl, n-hexyl and iso-hexyl. The C₁-C₆ alkyl may be C₁-C₄ alkyl.

The term “C₂-C₆ alkenyl” refers to a straight or branched group having 1 to 3 double bonds and 2 to 6 carbon atoms, such as, but not limited to, ethenyl, propenyl, isopropenyl, 1-butenyl, 2-butenyl, 2-methyl-1-propenyl, 1,3-butadienyl and 1,3,5-hexanetrienyl.

Where, Rⁿ¹ and Rⁿ² is each independently C₁-C₆ alkyl the dithiocarbamate within the compound of formula (I) may be referred to as a dialkyldithiocarbamate.

Preferably, each of Rⁿ¹ and Rⁿ² is C₂ alkyl such that the dithiocarbamate is a diethyldithiocarbamate, or each of Rⁿ¹ and Rⁿ² is C₁ alkyl such that the dithiocarbamate is a dimethyldithiocarbamate.

Most preferably, the dithiocarbamate is a diethyldithiocarbamate.

A dithiocarbamate anion may be generated from the compound of formula (I) in the presence of a metal, such as metal ion, in particular a metal selected from the group consisting of copper, zinc, platinum, iron, gold, silver and magnesium. The dithiocarbamate may act as bidentate ligand in coordination with the metal, forming a complex as described below. The chelation ability of the dithiocarbamate anion to the metal is dependent on maintaining the anionic form of the dithiocarbamate, which is only possible with the saccharides of the present invention. The anion may be generated under aqueous conditions.

According to formula (I), x is 1 or more, such as x is 1 or x is 2, 3, 4, 5, 6, 7, 8, 9 or 10. In one embodiment the value of x is equal to, or not more than, the number of saccharide units in the (poly)saccharide of group A.

Where the group A is a monosaccharide, then x may be 1.

In some embodiments, x is 1, such that there is one dithiocarbamate moiety present in the compound. In some preferred embodiments, x is 2, such that there are two dithiocarbamate moieties present in the compound.

Where x is 2 or more, this may provide an additional anti-cancer effect upon cleavage of the compound, as a plurality of dithiocarbamates may be made available from one compound of formula (I) for formation of a plurality of active complexes.

### Active Complex

A “complex” or “coordination complex” as described herein refers to the coordination of at least one organic compound with a single metal centre. Typically, the complex as described herein involves a combination of coordinate covalent bonds and/or ionic bonds between the organic compound and the metal centre.

An “active complex” as described herein refers to a complex or a coordination complex which may be generated in situ, such as in vivo or ex vivo, and is the species which provides the anti-cancer activity.

The mechanism for the cleavage of disulfiram to its monomeric diethyldithiocarbamate form in the presence of metal ions, and the subsequent formation of the copper coordination complex with the free diethyldithiocarbamate ligand is well known in the art (Lewis et al. Chem. Commun. 2014; 50; 13334-13337). The compound of formula (I) may be similarly cleaved to give a dithiocarbamate which coordinates to a metal, such as a metal ion.

Accordingly, there is provided a method of forming a complex, the method comprising contacting the compound of formula (I) with a metal, such as a metal ion, selected from the group consisting of copper, zinc, platinum, iron, gold, silver and magnesium, and preferably copper. The method may be performed in vivo or ex vivo. The method may be performed within a cell, such as a cancer cell.

The compound is cleaved in the presence of metal ions, typically metal ions in the +2 oxidation state, typically between the dithiocarbamate-saccharide M-S linkage, wherein M is metal and S is sulfur. Specifically, the compound is cleaved between a sulfur atom of a dithiocarbamate group and a carbon atom of the saccharide in the presence of metal ions. Typically, the carbon atom of the saccharide is the anomeric carbon atom of the (poly)saccharide. The compound of formula (I) may dissociate to form a complex and a (poly)saccharide cleavage product.

The complex may be a M(II) bis(N,N-dithiocarbamate) complex formed upon coordination of the bidentate dithiocarbamate ligand to a metal ion.

The M(II) bis(N,N-dithiocarbamate) complex is the active species for anti-cancer treatment, wherein M(II) is the metal in the +2 oxidation state. The M(II) bis(N,N-dithiocarbamate) complex may be a complex selected from the group consisting of Cu(II) bis(N,N-dithiocarbamate), Zn(II) bis(N,N-dithiocarbamate), Pt(II) bis(N,N-dithiocarbamate), Fe(II) bis(N,N-dithiocarbamate), Au(II) bis(N,N-dithiocarbamate), Ag(II) bis(N,N-dithiocarbamate) or Mg(II) bis(N,N-dithiocarbamate). Preferably, the complex is Cu(II) bis(N,N-dithiocarbamate) or Zn(II) bis(N,N-dithiocarbamate). More preferably, the complex is Cu(II) bis(N,N-diethyldithiocarbamate) or Zn(II) bis(N,N-diethyldithiocarbamate).

In some embodiments, the active specifies for anti-cancer treatment is a M(I) (N,N-dithiocarbamate) complex or a M(III) tris(N,N-dithiocarbamate) complex, wherein M(I) is the metal in the +1 oxidation state and M(III) is the metal in the +3 oxidation state, respectively.

In some embodiments, the compound of formula (I) comprises two or more dithiocarbamate groups, for example when x is 2 or more. In such embodiments, the compound is cleaved between a sulfur atom of a first dithiocarbamate and a carbon atom of the (poly)saccharide, and between a sulfur atom of a second dithiocarbamate and a carbon atom of the (poly)saccharide, in the presence of metal ions, such as copper ions. Additional cleavage steps occur between the dithiocarbamate groups the sulfur atom of the additional dithiocarbamates and the respective carbon atom of the (poly)saccharide.

Complete formation of the active complex may be determined by extraction of a precipitate following cleavage of the compound according to the method described in Lewis et al. Chem. Commun. 2014; 50; 13334. The active complex can be easily isolated and filtered from the reaction.

The saccharide cleavage product is derived from the group A, which is the (poly)saccharide connected to the thiocarbamate via a thioglycosidic bond. The saccharide cleavage product may therefore simply be the thiosaccahride corresponding to the group A, where the thioether of the saccharide that forms the connection to the thiocarbamate is formally replaced with hydroxyl.

The saccharide cleavage product may be selected from glucose, mannose, galactose, galactosylamine, xylose, 2-deoxy-glucose, 2-O-propyl-glucose, 2-N-acetyl-glucosamine, 2-N-acetyl-galactosamine, lactose, maltose, chitobiose, maltotriose, maltopentose and chitosan. Preferably, the saccharide cleavage product is a saccharide in the D-isomeric form. More preferably, the saccharide cleavage product is a saccharide 2-deoxy-D-glucose or chitosan.

As described herein, the phrase “in the presence of” refers to metal, such as metal ions, being within the vicinity of the compound such that the compound is capable of being cleaved and dissociating into the bidentate dithiocarbamate ligand and the saccharide cleavage product by reacting the compound with the metal, such as the metal ion. For example, the copper ions may be in solution with the compound, such that it is in contact with the compound, and so would be regarded as “in the presence of”. The cleavage mechanism is analogous to the cleavage mechanism of disulfiram with copper ions (Lewis et al. Chem. Commun. 2014; 50; 13334).

### Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

For the compound, a salt may be formed with a suitable anion by reacting the amine portion of the dithiocarbamate or a hydroxyl of the saccharide with a suitable inorganic acid.

Unless otherwise specified, a reference to a particular compound also includes salt forms thereof.

### Pharmaceutical Composition

A particularly important second aspect of the present invention is directed to a pharmaceutical composition comprising the compound of formula (I) and a pharmaceutically acceptable carrier, and the use of the composition in methods of treating cancer.

The pharmaceutical composition provided herein may comprise the compound together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

The pharmaceutical composition provided herein may include one or more excipients, e.g., solvents, solubility enhancers, suspending agents, buffering agents, isotonicity agents, chemical permeability enhancers, targeting moieties, antioxidants or antimicrobial preservatives. When used, the excipients of the compositions will not adversely affect the stability, bioavailability, safety, and/or efficacy of the active ingredients, i.e. the complex formed from the compound in the presence of metal. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

The pharmaceutical composition provided herein may be prepared using a pharmaceutically acceptable “carrier” composed of materials that are considered safe and effective.

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

In the present invention, the carrier of the pharmaceutical composition may be both to assist in the delivery of the compound to the target cancer cell and to prevent the formation of the active complex during the delivery of the composition until the target cancer cell is reached. The latter is because the active complex and the subsequent cleavage product each have individually relatively poor pharmacokinetics compared to the compound. Therefore, the compound is advantageous in that it provides good delivery to the target cancer cell without being metabolised before it reaches the target cancer cell.

It is well known that the concentration of some metals, such as copper, zinc, platinum, iron, gold, silver and magnesium, is raised in cancer cells to promote growth through processes such as angiogenesis, thereby also providing a targeted area of the body for delivery of the compound.

It may be appropriate to administer a metal ion as a supplement to a patient, especially to a patient who has a metal deficiency, such as a copper deficiency, or who for some other unrelated reason lacks sufficient metal accumulation in cancer tissue, to assist in the delivery of the compound.

From the point of view of anti-cancer activity, it is most preferable to administer a copper ion to a patient. However other metal ions, such as a zinc ion, may also be suitable.

Therefore, in view of providing improved anti-cancer activity, it is more preferable to only allow the formation of the complex as the active ingredient once the target cancer cell is reached. Accordingly, there is a provided a pharmaceutical composition comprising the compound and a pharmaceutically acceptable carrier or excipient, optionally comprising a saline or a buffered saline. The pharmaceutically acceptable carrier or excipient may be able to hold the compound within itself, such that the compound may be protected from cleavage in the presence of copper. Preferably, the pharmaceutically acceptable carrier or excipient may be able to encapsulate the compound within itself.

The use pharmaceutically acceptable carrier or excipient is preferable to increase lipophilicity, thus increasing the permeability of the compound across a cellular membrane in order to provide a targeted delivery of the compound to cancer cells. Further, a pharmaceutically acceptable carrier or excipient is preferable to protect the compound from premature cleavage with metal ions until the target cancer cell is reached.

Different methods for preparing a formulation for disulfiram and disulfiram derivatives using such carriers are known in the art. Preferably, the carrier is a liposome. For example, a liposome may contain copper within the aqueous core (Wehbe et al. Int. J Nanomedicine. 2017; 12; 4129-4146). In such an example, the liposome may be loaded with copper in the form of a copper salt, such as copper sulfate or copper gluconate, in the hydrophilic core and incubated with diethyldithocarbamates in a buffer solution. Subsequently, the formation of the copper complex may be determined over a 60-minute incubation period. Preferably, the liposome comprises phospholipids for the purposes of increasing the permeability of the compound across a cellular membrane. Preferably, the polymeric micelle, or nanoparticles or microparticles, is selected from the group consisting of poly(lactic-co-glycolic acid) and polycaprolactone.

While carriers such as liposomes or other nanoparticles or microparticles may not be necessary for the purposes of solubility and stability, such a carrier may be useful in instances where it is preferable to provide a formulation of the compound and copper together in the pharmaceutical composition for the purposes of a simultaneous administration.

As an alternative to providing a carrier for the pharmaceutical composition for the purposes of the prevention of copper complexation, a further aspect of the present invention is a kit comprising a pharmaceutical composition comprising the compound of general formula (I) and a metal, such as a metal ion, which provides a route of independent administration of the two components to a subject with cancer.

Accordingly, the pharmaceutical composition may be administered into a subject with cancer. The metal, such as a metal ion, may be optionally administered into the subject if deemed necessary, and is particularly useful as a supplement for the purposes of targeting and delivery of the compound to the cancer tissue.

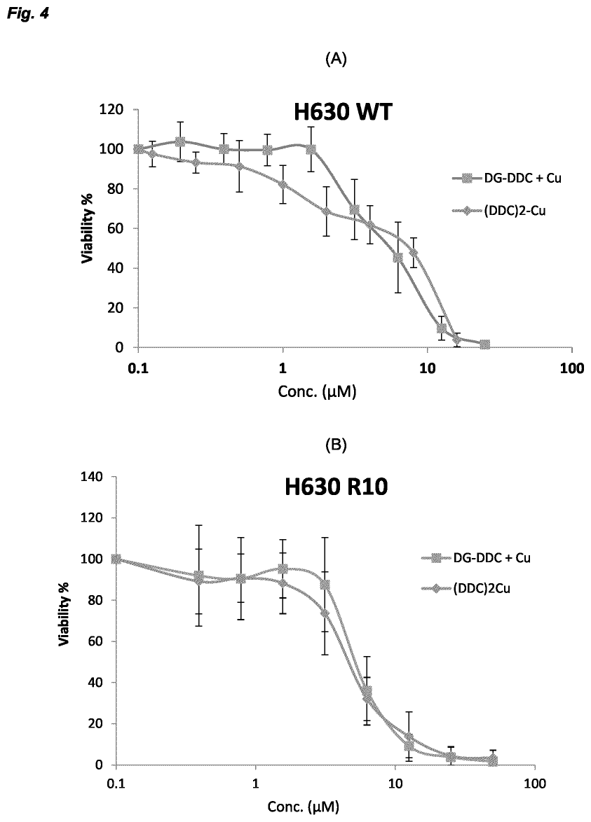

The compound or pharmaceutical composition may be administered in combination with other anti-cancer drugs in the method of treatment of a subject with cancer. Examples of known anti-cancer drugs include fluorouracil (5FU). In this connection, FIG. 4C shows the synergic cytotoxic effect between the combination of 2-deoxy-glycosyl diethyldithiocarbamate (DG-DDC) with copper(II) (10 μM) and fluorouracil (5FU) in comparison with the individual cytotoxic effect of 2-deoxy-glycosyl diethyldithiocarbamate (DG-DDC) with copper(II) (10 μM) and individual cytotoxic effect of fluorouracil (5FU).

### Metals

As described herein, a metal is provided as a metal supplement to facilitate in the cleavage of the compound to produce the active complex. The metal may be provided in situ within the pharmaceutical composition comprising the compound, or the metal may be provided separately from the pharmaceutical composition as a kit for the purposes of independent administration. The metal may be preferable for treating a subject with cancer who has a metal insufficiency in their cancer tissue, such as in the case where the subject has a copper deficiency.

The metal may be in the +1 or +2 oxidation state, such that a metal salt may comprise the metal and an anion. Preferably, the metal is in the +2 oxidation state. The metal salt may be selected from the group consisting of metal sulfate, metal chloride, metal hydroxide, metal nitrate, metal oxide, metal acetate, metal fluoride, metal bromide, metal carbonate, metal carbonate hydroxide, metal chlorate, metal arsenate, metal azide, metal acetylacetonate, metal aspirinate, metal cyanaurate, metal glycinate, metal phosphate, metal perchlorate, metal selenite, metal sulfide, metal thiocyanate, metal triflate, metal tetrafluoroborate, metal acetate triarsenite, metal benzoate, metal arsenite, metal chromite, metal gluconate, metal peroxide and metal usnate.

The metal, such as metal ion, may be selected from the group consisting of copper, zinc, platinum, iron, gold, silver and magnesium. Preferably, the metal is copper.

The metal may be provided as a component within a solid or in aqueous solution. The skilled person would understand that, where the metal is a solid, they may either administer the solid metal to a subject with cancer for solid administration, such as in the form of a pill or tablet, or they may dissolve the solid metal in aqueous solution to produce an aqueous metal solution for liquid administration.

The metal formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The metal formulation may be prepared to provide for rapid or slow release; immediate, delayed, timed, or sustained release; or a combination thereof.

Metal formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses. Preferably, the copper formulation is a tablet, pill or capsule.

A metal formulation as a tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

### Cancers

The invention relates to methods for the treatment of cancer in subjects. A “cancer” is defined as a genetic disease in which certain cells within the body grow uncontrollably and spread to other parts of the body, caused by changes in the genes which control cell function. A “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chrondrosarcoma, osteosarcoma).

According to the National Cancer Institute, the most common types of cancers are, in order of descending new cases in 2020, breast cancer, lung and bronchus cancer, prostate cancer, colorectal cancer, melanoma of the skin, bladder cancer, non-Hodgkin lymphoma, kidney and renal pelvis cancer, endometrial cancer, leukaemia, pancreatic cancer, thyroid cancer, and liver cancer.

Methods of treatment that are regarded as “anti-cancer” are those procedures which include radiation and chemical agents used to attack cancer tumour cells, whereas “anti-carcinogen” treatment relates to the use of chemical agents which work against the processes that may lead to cancer, such as agents that act as antioxidants, and essential substances that help the immune, hormonal, and other systems to prevent carcinogenesis.

Some sugar-linked dithiocarbamates (using glucose, cellobiose and lactose as the sugar) have been previously disclosed as anti-carcinogenic compounds (Gopalaswamy et al. Anticancer Res. 1998; 18 (3A); 1827-1832), including those which also have improved solubility (Lee et al. J Med Chem. 1994; 37 (19); 3154-3162). It will be appreciated that these compounds were only described as for preventing or inhibiting processes which lead to cancer formation, rather for treating a patient who already has cancer. Specifically, there is an absence of the disclosure of the method of treatment of a patient with cancer. Furthermore, it is believed by the present inventor that there is no definitively conclusive confirmation of a high stability of the compounds as no stability studies were conducted in serum or in blood and, while in vivo studies were present, they were nonetheless not statistically nor analytically validated.

In one embodiment of the invention, the group A is not glucose, cellobiose and lactose connected via a thioglycosidic bond.

Accordingly, the first aspect of the present invention is the compound of formula (I) for use in the treatment of a subject with cancer. The compound shows excellent anti-cancer activity against a number of cancer cell lines as described herein.

Preferably, the cancer to be treated of the present invention is colorectal cancer, breast cancer, lung cancer or brain cancer.

A type of colorectal cancer includes colorectal adenocarcinoma, such as colorectal adenocarcinoma having a cell type selected from the group consisting of the following cell lines: C10, C125PM, C2BBE1, C75, C80, C84, C99, CACO2, CAR1, CCK81, CL11, CL14, CL34, CL40, COLO201, COLO205, COLO320, COLO320HSR, COLO678, CW2, DIFI, DLD1, ECC4, GEO, GISTT1, GP2D, GP5D, HCC2998, HCC56, HCT116, HCT15, HCT8, HRT18, HT115, HT29, HT55, JVE127, KM12, LOVO, LS1034, LS123, LS180, LS411N, LS513, MDST8, NCIH508, NCIH630, NCIH684, NCIH716, NCIH747, OUMS23, RCM1, RKO, SKCO1, SNU1033, SNU1040, SNU1197, SNU1544, SNU175, SNU283, SNU407, SNU503, SNU61, SNU81, SNUC1, SNUC2A, SNUC2B, SNUC4, SNUC5, SW1116, SW1417, SW1463, SW403, SW48, SW480, SW527, SW620, SW626, SW837, SW948, T84, TGBC18TKB and TT1TKB.

Colorectal cancers for treatment include primary colorectal lymphomas, gastrointestinal stromal tumors, leiomyosarcomas, carinoid tumors and melanomas.

A type of breast cancer includes invasive ductal carcinoma, such as invasive ductal carinoma having a cell type selected from the group consisting of the following cell lines: BT549, CAL120, CAL51, HCC1395, HMC18, HS578T, MDAMB157, MDAMB231, MDAMB436, SUM149PT and SUM159PT.

A type of lung cancer includes non-small cell lung cancer (NSCLC), such as NSCLC adenocarcinoma having a cell type selected from the group consisting of the following cell lines: 201T, A427, A549, ABC1, CALU3, COLO699, CORL105, DV90, EKVX, GLC82, HCC1171, HCC1833, HCC2108, HCC2279, HCC2935, HCC364, HCC4006, HCC44, HCC461, HCC515, HCC78, HCC827, HCC827GR5, HLC1, HOP62, JHU028, LC2AD, LXF289, MORCPR, NCIH1355, NCIH1373, NCIH1395, NCIH1435, NCIH1437, NCIH1563, NCIH1568, NCIH1573, NCIH1623, NCIH1648, NCIH1650, NCIH1651, NCIH1666, NCIH1693, NCIH1734, NCIH1755, NCIH1781, NCIH1792, NCIH1793, NCIH1819, NCIH1838, NCIH1944, NCIH1975, NCIH1993, NCIH2009, NCIH2023, NCIH2030, NCIH2073, NCIH2085, NCIH2087, NCIH2122, NCIH2126, NCIH2228, NCIH2291, NCIH23, NCIH2342, NCIH2347, NCIH2405, NCIH3122, NCIH322, NCIH322M, NCIH3255, NCIH358, NCIH441, NCIH522, NCIH650, NCIH838, NCIH854, PC14, PC3JPC3, PC9, RERFLCAD1, RERFLCAD2, RERFLCKJ, RERFLCMS, SKLU1, SW1573 and VMRCLCD.

A type of brain cancer includes glioblastoma, such as astrocytoma having a cell type selected from the group consisting of the following cell lines: 1321N1, 42MGBA, 8MGBA, CCFSTTG1, KINGS1, KS1, LN235, LN319, LNZTA3WT4, SF126, SF767, SKMG1, SW1088, SW1783, U118MG, U251MG, U251MGDM and U87MG.

Preferably, the colorectal cancer is adenocarcinoma, the breast cancer is invasive ductal carcinoma, the lung cancer is non-small cell lung cancer and the brain cancer is glioblastoma.

Most preferably, the colorectal cancer is the H630 cell line, the breast cancer is the MDA-MB-231 cell line or the MCF7 cell line, the lung cancer is the A549 cell line, and the brain cancer is the U87MG cell line.

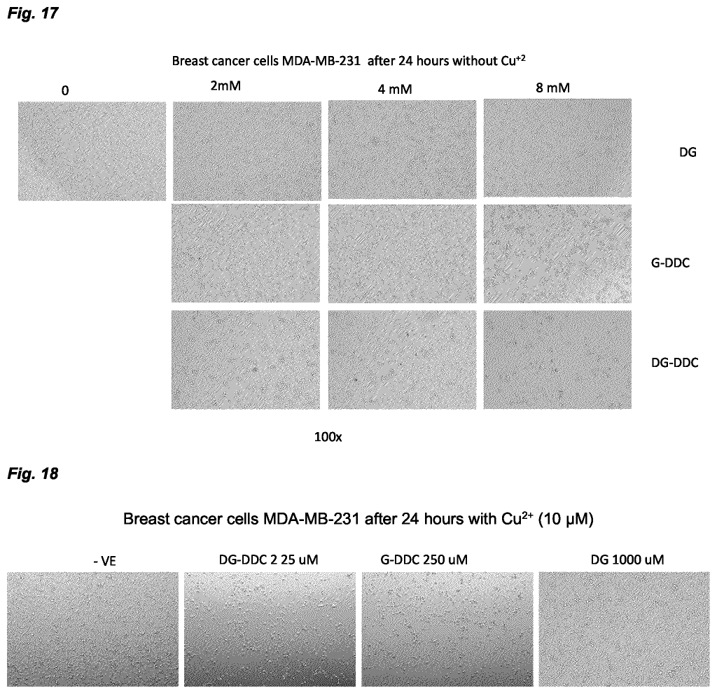

FIG. 16 shows microscopy images of lung cancer cells A549 after 24 hours in 2-deoxy-glucose, glycosyl diethyldithiocarbamate and 2-deoxy-glycosyl diethyldithiocarbamate without copper(II) and FIG. 17 shows microscopy images of breast cancer cells

MDA-MB-231 after 24 hours in 2-deoxy-glucose, glycosyl diethyldithiocarbamate and 2-deoxy-glycosyl diethyldithiocarbamate without copper(II). For both lung cancer cells and breast cancer cells, 2-deoxy-glucose shows slight toxicity effect on cancer cells at high concentration (8 mM) with the cells started to detach from the surrounding. However, a similar effect is shown by glycosyl diethyldithiocarbamate at lower concentration (4 mM) with signs of apoptosis shown at 8 mM (shrinkage, cell detachment). A more toxic effect is shown by 2-deoxy-glycosyl diethyldithiocarbamate compared to glycosyl diethyldithiocarbamate at similar concentrations.

FIG. 18 shows microscopy images of breast cancer cells MDA-MB-231 after 24 hours in 2-deoxy-glucose, glycosyl diethyldithiocarbamate and 2-deoxy-glycosyl diethyldithiocarbamate with copper(II). In the presence of copper(II), 2-deoxy-glycosyl diethyldithiocarbamate and glycosyl diethyldithiocarbamate showed a high increase in their cytotoxicity. The 2-deoxy-glycosyl diethyldithiocarbamate showed apoptotic signs (shrinkage, cell detachment, membrane blebbing, ultrastructural modification of cytoplasmic organelles and a loss of membrane integrity) at 25 μM after 24 hours of incubation, whereas glycosyl diethyldithiocarbamate showed similar effect at higher concentration (250 μM). Cells incubated with 2-deoxy-glucose at 1000 μM showed no features of apoptosis.

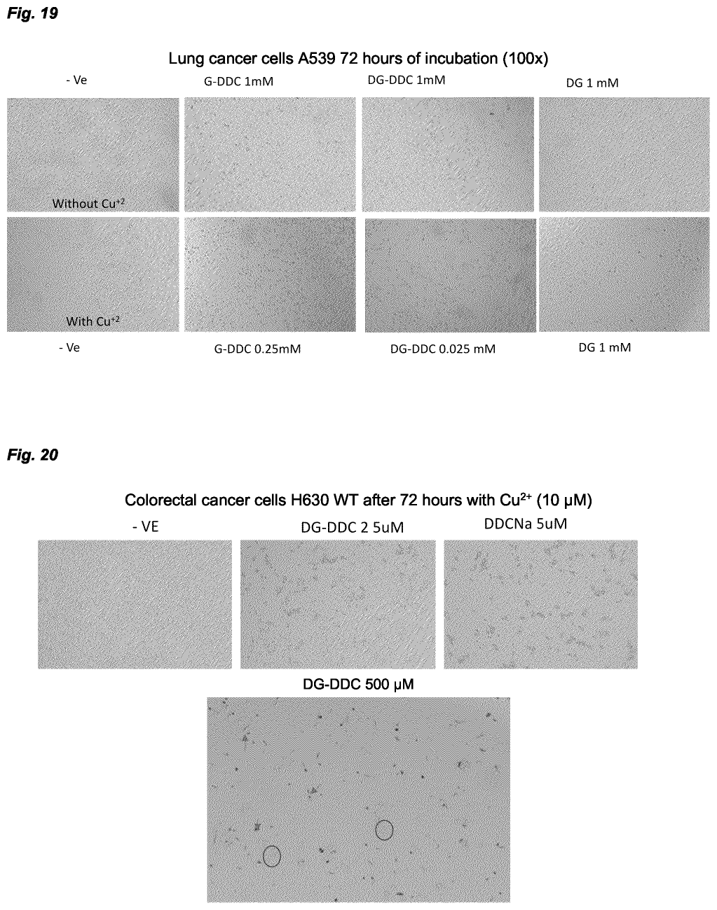

FIG. 19 shows microscopy images of lung cancer cells A549 after 72 hours in 2-deoxy-glucose, glycosyl diethyldithiocarbamate and 2-deoxy-glycosyl diethyldithiocarbamate with and without copper(II) (10 μM). Microscopy images taken 72 h after treatment showing the negative controls with and without copper(II) are similar, glycosyl diethyldithiocarbamate and 2-deoxy-glycosyl diethyldithiocarbamate are cytotoxic against lung cancers cells but the cytotoxicity is boosted by adding copper and 2-deoxy-glucose has no cytotoxic effect at 1 mM concentration with or without copper(II).

FIG. 20 shows microscopy images of colorectal cancer cells H630 WT after 72 hours in 2-deoxy-glycosyl diethyldithiocarbamate and sodium diethyldithiocarbamate with copper(II).

Similar effect was shown by 2-deoxy-glycosyl diethyldithiocarbamate and sodium diethyldithiocarbamate at the same concentrations (5 μM) in presence of copper(II) (10 μM). Furthermore, colorectal cancer cells with 2-deoxy-glycosyl diethyldithiocarbamate (500 μM) and copper(II) (10 μM) show Cu(II) bis(N,N-diethyldithiocarbamate) complex crystals, as indicated by the arrows, and cell debris, as indicated by the circles.

Subject with Cancer

A subject with cancer is a subject who, at the time of the administration of the compound or pharmaceutical composition comprising the compound, currently has cancer. That is, a subject with cancer is a subject whose body contains cells which are already growing abnormally outside of the normal functions of the body, and may be professionally confirmed by cancer diagnosis, such as analysis using computerised tomography (CT) scan, bone scan, magnetic resonance imaging (MRI), positron emission tomography (PET) scan, ultrasound or X-ray.

The subject to be treated may be any animal or human. The subject is preferably a non-human mammal, more preferably mammalian, most preferably a human subject. The subject may be male or female. Therapeutic uses may be in human or animals (veterinary use).

When the subject is a human subject, the human subject may be a patient who is diagnosed with cancer, the diagnosis preferably being done by a medical professional.

It should be apparent to a medical professional whether to administer the pharmaceutical composition simultaneously with a metal supplement to a patient, to administer the pharmaceutical composition separately from a metal supplement to a patient, or to administer the pharmaceutical composition without a metal to a patient. This should be apparent to the medical professional depending on the particular requirements and characteristics of the patient.

It should be apparent that a subject with cancer is different to a subject at risk of cancer, such as a patient at risk of cancer. A subject at risk of cancer is a subject who may have a significantly higher probability of getting cancer than an average individual, perhaps due to certain underlying health conditions, but nevertheless does not yet have cancer at the time of the administration of the compound or pharmaceutical composition comprising the compound.

Accordingly, a further aspect is a method of treatment of a subject with cancer, the method comprising the step of an intravenous administration of the compound of formula (I) or the pharmaceutical composition.

In some embodiments, the method of treatment further comprises the step of an administration of a metal, such as a metal ion, selected from the group consisting of copper, zinc, platinum, iron and magnesium, and preferably copper, including the ions thereof.

In some embodiments, the method of treatment further comprises the step of an intravenous administration of a pharmaceutical composition.

A further aspect is a method of treatment of a subject with cancer, wherein the pharmaceutical composition for use is administered together with at least one other anti-cancer drug.

### Other Preferences

Methods according to the present invention may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism.

Where the method is performed in vitro it may comprise a high throughput screening assay. Test compounds used in the method may be obtained from a synthetic combinatorial peptide library, or may be synthetic peptides or peptide mimetic molecules. Other test compounds may comprise defined chemical entities, oligonucleotides or nucleic acid ligands.

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### EXAMPLES

### Example 1: Glycosyl diethyldithiocarbamate (G-DDC)

Glycosyl diethyldithiocarbamate (G-DDC) was synthesised and characterised as reported before by Li et al. Tetrahedron Letters, 2016; 57 (31) 3529-3531.

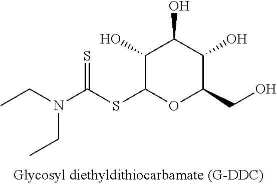 [SMILES: CCN(CC)C(=S)SC1O[C@H](CO)[C@@H](O)[C@H](O)[C@H]1O]

### Mtt Assay (Cytotoxicity Against Cancer Cells)

The H630 WT (passage 12-21) and H630 R10 (passage 4-13) cells were seeded in 96-well plates at seeding density of 1×10⁴ cells/well in Dulbecco's modified Eagle's medium (DMEM) with 10% FBS, 1 mM sodium pyruvate, 2 mM L-glutamine and 0.1 mM non-essential amino acids. Cells were incubated at 37° C., 5% CO₂ and 95% relative humidity. Cells were constantly exposed to different concentrations of the tested compound in combination with 10 μM of CuCl₂ for 72 h and then subjected to a standard 3-(4,5-Dimethylthiazol-2-yl)-2,5-Diphenyltetrazolium bromide (MTT) assay. The experiments were carried out in triplicates and the IC₅₀ values were calculated (see FIG. 2).

### In Vitro Biostability (Stability in Horse Serum)

A sample 0.5 ml of 1 mmol G-DDC was preheated at 37° C., added to 2 mL of horse serum (preheated at 37° C.) and incubated in a shaking water bath at 37° C. (Grant OLS Aqua Pro, Shepreth, UK) and 100 rpm. For free DS, 25 μL of 4 mg/mL DS in DMSO was pipetted in to 2 mL of horse serum diluted with 475 UL of distilled water (preheated at 37° C.). At specific time intervals, aliquots of 200 μL were added to 500 μL of acetonitrile and vortexed for 10 second. The mixture solution was centrifuged at 10,000×g for 10 min (Heraeus Fresco 17, UK). The supernatant “A” was collected, and the pellet was re-suspended in 0.5 methanol vortex for 30 s and heated in the water bath 37° C. for 2 min, then vortexed for 10 s again and centrifuged at 10,000×g for 10 min. The supernatant “B” was collected, added to supernatant “A” and analysed by HPLC. For the control, the same concentrations and previous steps were followed, but by replacing the horse serum with distilled water. The control was used as 100% for the stability calculations (see FIG. 1).

### Example 2: G-DDC and Copper

An aqueous solution of G-DDC (1 mM) was added to CuCl₂ solution (1 mM). at time intervals, 50 μl of the combination mixture was withdrawn and transferred to Spin X centrifuge tube with cellulose acetate filter (0.45 μm) containing 450 μL HPLC water and centrifuged for 10 min at 13,000 rpm.

Samples then analysed by HPLC. HPLC methods: UltiMate 3000 UHPLC (Thermo Fisher Scientific, Loughborough, UK) with Accucore150-C₁₈ column, 2.1×100 mm (Thermo Fisher Scientific, Loughborough, UK) were used. The mobile phase comprised 90% HPLC-grade ACN and 10% HPLC-grade zinc sulphate buffer (10-10 mol/L). The flow rate was 0.1 mL/min, and UV detection was performed at 260 nm with an injection volume of 10 μL.

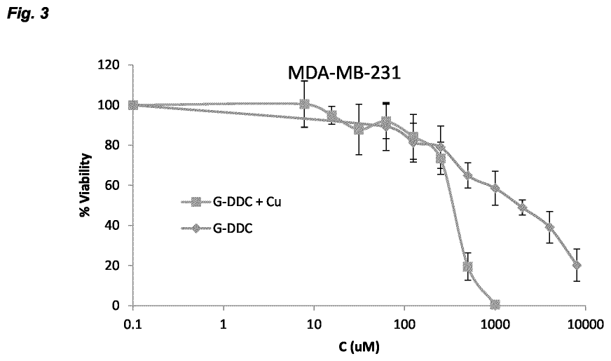

Cytotoxicity against cancer cells were analysed using the same method as that of Example 1, and results are shown in FIGS. 2, 3 and 6.

### Example 3:2-Deoxy-glycosyl diethyldithiocarbamate (DG-DDC)

2-Deoxy-glycosyl diethyldithiocarbamate (DG-DDC) was synthesised and characterised using analogous methods to Li et al. Tetrahedron Letters, 2016; 57 (31) 3529-3531, except 2-deoxy-glucose was used as the saccharide.

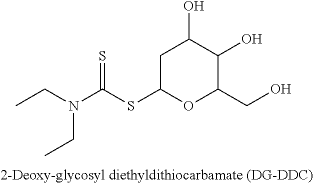 [SMILES: CCN(CC)C(=S)SC1CC(O)C(O)C(CO)O1]

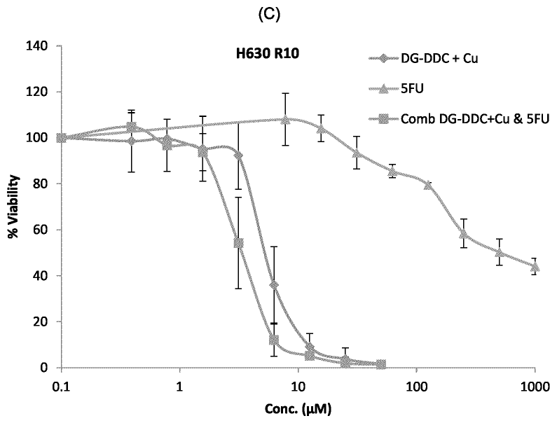

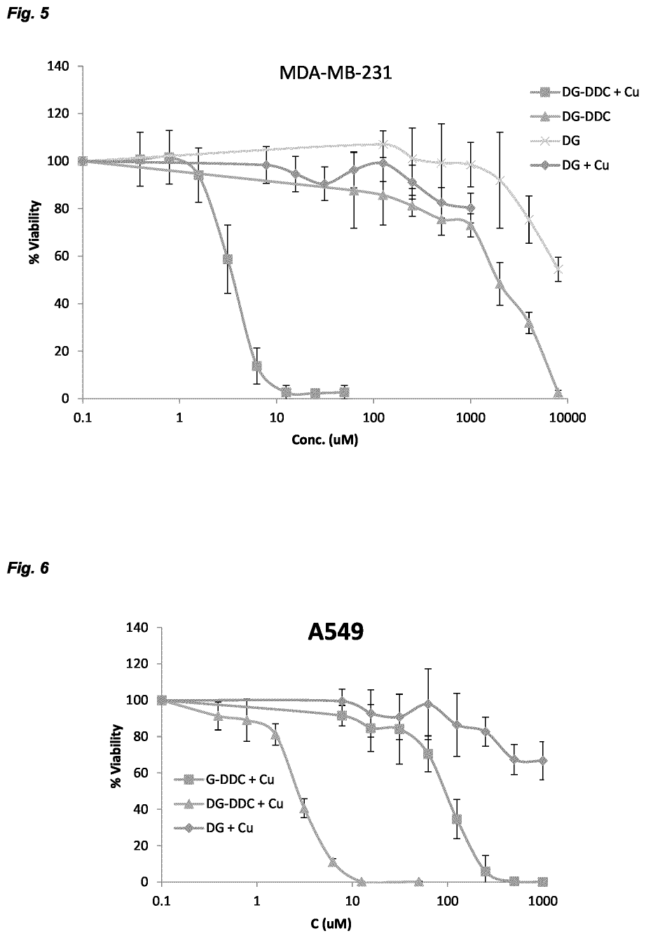

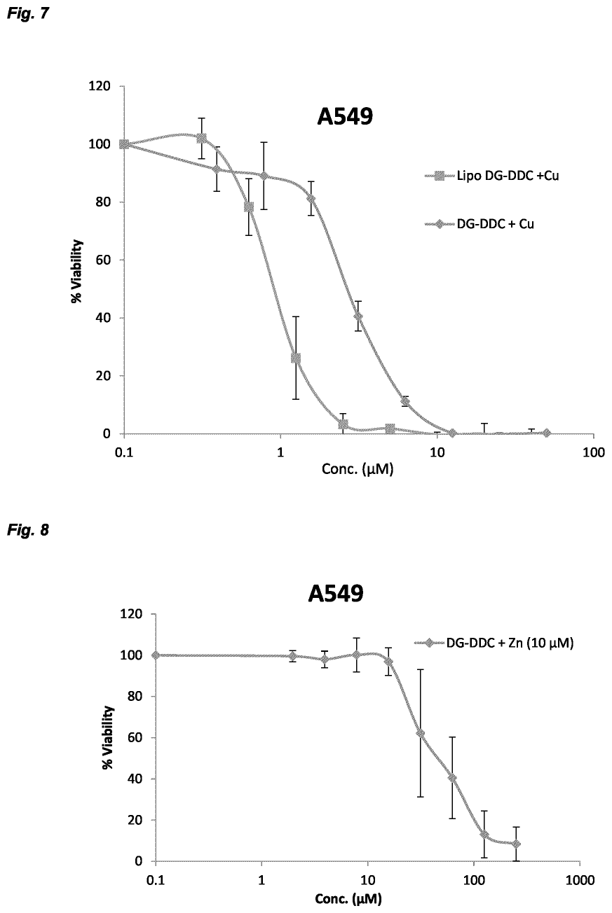

In vitro biostability (stability in horse serum) and cytotoxicity against cancer cells were analysed using the same method as that of Example 1, and results are shown in FIG. 1, and FIGS. 5 and 7, respectively.

### Example 4: DG-DDC and Copper

An aqueous solution of DG-DDC (1 mM) was added to CuCl₂ solution (1 mM). at time intervals, 50 μl of the combination mixture was withdrawn and transferred to Spin X centrifuge tube with cellulose acetate filter (0.45 μm) containing 450 μL HPLC water and centrifuged for 10 min at 13,000 rpm.

Samples then analysed by HPLC. HPLC methods: UltiMate 3000 UHPLC (Thermo Fisher Scientific, Loughborough, UK) with Accucore150-C18 column, 2.1×100 mm (Thermo Fisher Scientific, Loughborough, UK) were used. The mobile phase comprised 90% HPLC-grade ACN and 10% HPLC-grade zinc sulphate buffer (10-10 mol/L). The flow rate was 0.1 mL/min, and UV detection was performed at 260 nm with an injection volume of 10 μL.

Cytotoxicity against cancer cells were analysed using the same method as that of Example 1, and results are shown in FIGS. 4, 5, 6 and 7.

### Example 5: Liposomal Formulation of DG-DDC

Liposomes have previously been used as a drug delivery system, especially for anti-cancer drugs. DG-DDC loaded liposomes were prepared following the methods reported by Najlah et al. Pharmaceutics. 2019; 11 (11); 610 with some modifications:

The lipid phase (HSPC: Ch; 1:1 mole ratio) (50 mg) was dissolved in absolute ethanol (70 μL) within a glass vial which then capped and bath-sonicated (Elmasonic P30H Ultrasonic Bath, UK) at 37 kHZ, 70° C. for 10 mins. DG-DDC from Example 3 was added to the aqueous. Aqueous (water) phase (5 mL), heated significantly above the Tm of the lipid, was added to the lipid phase above followed by continuous sonication at 37 kHZ, 70° C. for one hour. Immediately following sonication, samples were allowed to equilibrate at room temperature for at least two hours before any further experimental procedures took place. The same steps above were applied without the addition of DG-DDC to create blank (empty) formulation. Both empty and loaded formulations were made in triplicates (n=3).

Liposomes were characterised as follows: the size, polydispersity and surface charge of the liposomes were analysed by recording the hydrodynamic diameter (Z_{average}) and polydispersity index (PI) and Zeta potential respectively, using the Zetasizer instrument (Zetasizer nano, Malvern Instruments Ltd., Malvern, UK). The liposomal formulation of DG-DDC has the following characteristics: size (Z_{average}): 78.9±14.3 nm, and Zeta potential: −9.4±0.7 mV.

Cytotoxicity against cancer cells was analysed using the same method as that of Example 1, and results are shown in FIG. 7.

### Example 6: DG-DDC and Zinc

Cytotoxicity against cancer cells were analysed using the same method as that of Examples 1 and 4, but CuCl₂ was replaced by ZnCl₂ (10 μM) and results are shown in FIG. 8.

### Example 7: 2-N-acetyl-glycosylamine diethyldithiocarbamate (AG-DDC)

2-N-acetyl-glycosylamine diethyldithiocarbamate (AG-DDC) was synthesised and characterised using analogous methods to Li et al. Tetrahedron Letters, 2016; 57 (31) 3529-3531, except 2-N-acetyl-glycosamine was used as the saccharide.

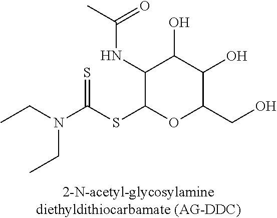 [SMILES: CCN(CC)C(=S)SC1OC(CO)C(O)C(O)C1NC(C)=O]

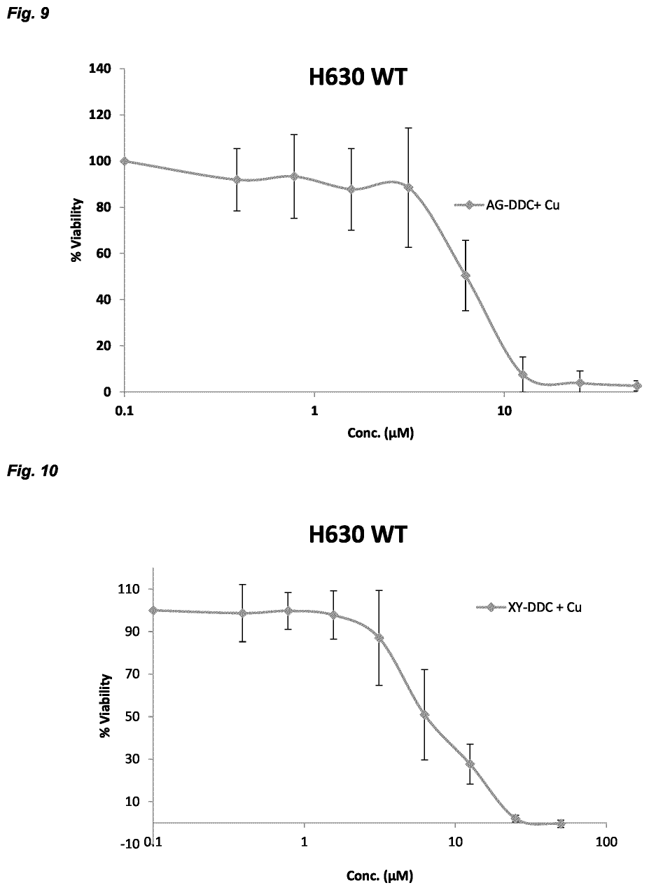

In vitro biostability (stability in horse serum) and cytotoxicity against cancer cells were analysed using the same method as that of Example 1, and results are shown in FIG. 1 and FIG. 9, respectively.

### Example 8: Xylosyl diethyldithiocarbamate (XY-DDC)

Xylosyl diethyldithiocarbamate (XY-DDC) was synthesised and characterised using analogous methods to Li et al. Tetrahedron Letters, 2016; 57 (31) 3529-3531, except xylose was used as the saccharide.

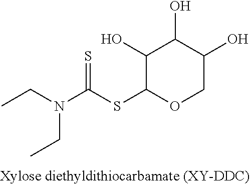 [SMILES: CCN(CC)C(=S)SC1OCC(O)C(O)C1O]

Cytotoxicity against cancer cells was analysed using the same method as that of Example 1, and results are shown in FIG. 10.

### Additional Examples

### Example 9: Lactosyl diethyldithiocarbamate (La-DDC)

Lactosyl diethyldithiocarbamate (La-DDC) was synthesised and characterised using analogous methods to Li et al. Tetrahedron Letters, 2016; 57 (31) 3529-3531, except lactose was used as the saccharide.

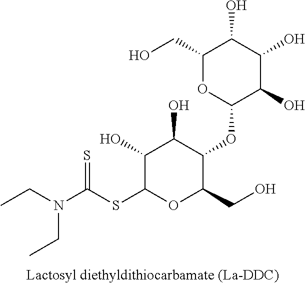 [SMILES: CCN(CC)C(=S)SC1O[C@H](CO)[C@@H](O[C@@H]2O[C@H](CO)[C@H](O)[C@H](O)[C@H]2O)[C@H](O)[C@H]1O]

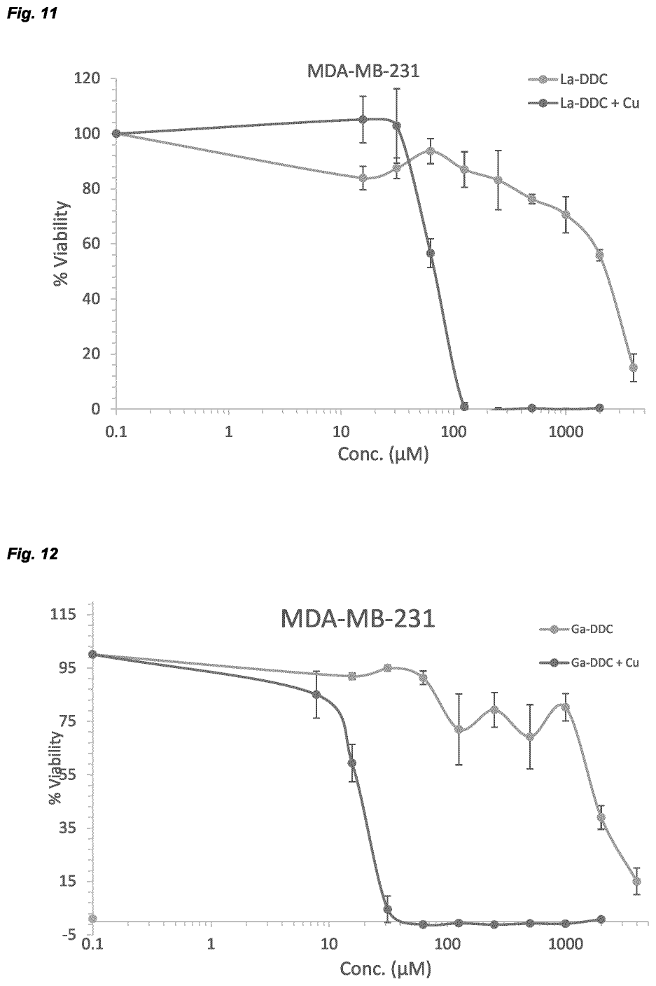

Cytotoxicity against cancer cells were analysed using the same method as that of Example 1, and results are shown in FIG. 11.

### Example 10: Galactosyl diethyldithiocarbamate (Ga-DDC)

Galactosyl diethyldithiocarbamate (Ga-DDC) was synthesised and characterised using analogous methods to Li et al. Tetrahedron Letters, 2016; 57 (31) 3529-3531, except galactose was used as the saccharide.

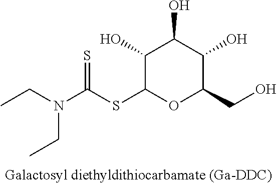 [SMILES: CCN(CC)C(=S)SC1O[C@H](CO)[C@@H](O)[C@H](O)[C@H]1O]

Cytotoxicity against cancer cells were analysed using the same method as that of Example 1, and results are shown in FIG. 12.

### Example 11: Mannosyl diethyldithiocarbamate (Ma-DDC)

Mannosyl diethyldithiocarbamate (Ma-DDC) was synthesised and characterised using analogous methods to Li et al. Tetrahedron Letters, 2016; 57 (31) 3529-3531, except mannose was used as the saccharide.

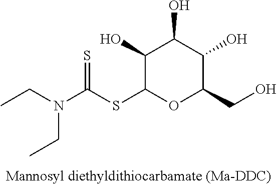 [SMILES: CCN(CC)C(=S)SC1O[C@H](CO)[C@@H](O)[C@H](O)[C@@H]1O]

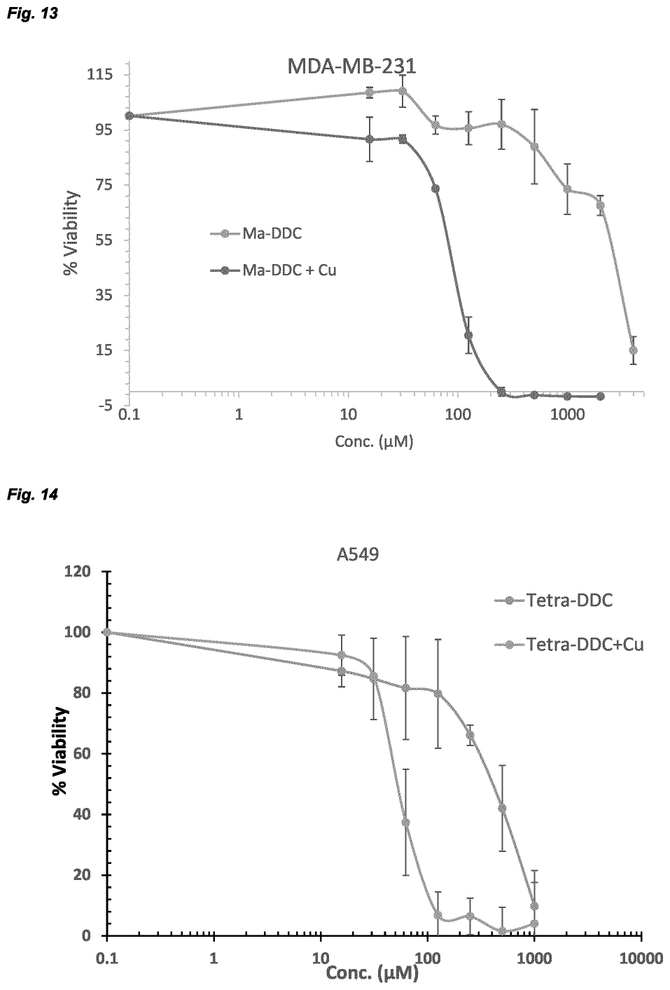

Cytotoxicity against cancer cells were analysed using the same method as that of Example 1, and results are shown in FIG. 13.

### Example 12:3-D-Glucopyranose, 1-thio-, 2,3,4,6-tetraacetate 1-(N,N-diethylcarbamodithioate) (Tetra-DDC)

Tetra-DDC was purchased from Alfa Chemistry, Ronkonkoma, NY 11779-7329, USA.

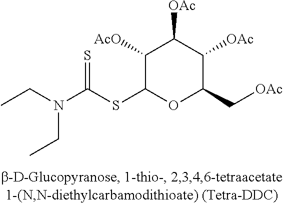 [SMILES: CCN(CC)C(=S)SC1O[C@H](COC(C)=O)[C@@H](OC(C)=O)[C@H](OC(C)=O)[C@H]1OC(C)=O]

Cytotoxicity against cancer cells were analysed using the same method as that of Example 1, and results are shown in FIG. 14.

### Example 13: DG-DDC and Copper in Combination with Fluorouracil (5FU)

DG-DDC and copper were prepared using the same method as that of Example 4. 5FU was purchased from Sigma Aldrich, Dorset, UK. Equivalent molar ratios of DG-DDC and copper (Example 4) and 5FU were prepared for the Example 13.

Cytotoxicity against cancer cells were analysed using the same method as that of Example 1, and results are shown in FIG. 4C.

TABLE 3
IC₅₀ values for different cancer cell lines for Examples 1 to 8.

| Example | Compound | H630 WT | H630 R10 | MDA-MB-231 | A549 |
|---|---|---|---|---|---|
| 1 | G-DDC (μM) | | | 2178 ± 356.1 | |
| 2 | G-DDC + Cu (μM) | 127 ± 13.2 | | 347.5 ± 44.7 | 95.9 ± 21.3 |
| 3 | DG-DDC (μM) | | | 2055 ± 54.70 | |
| 4 | DG-DDC + Cu (μM) | 5.2 ± 1.7 | 5.3 ± 0.9 | 3.62 ± 0.22 | 2.79 ± 0.25 |
| 5 | Lipo. DG-DDC + Cu (μM) | 1.5 ± 0.2 | | | 0.94 ± 0.12 |
| 6 | DG-DDC + Zn (μM) | | | | 49.6 ± 18.6 |
| 7 | AG-DDC + Cu (μM) | 5.8 ± 1.2 | | | |
| 8 | XY-DDC + Cu (μM) | 6.9 ± 2.6 | | | |
| 9 | La-DDC + Cu (μM) | | | 89.7 ± 12.6 | |
| 10 | Ga-DDC + Cu (μM) | | | 17.7 ± 8.2 | |
| 11 | Ma-DDC + Cu (μM) | | | 78.6 ± 12.6 | |
| 12 | Tetra-DDC + Cu (μM) | | | | 53.4 ± 11.5 |
| 13 | DG-DDC + Cu + 5FU (μM) | | 3.6 ± 0.7 | | |

TABLE 4
IC₅₀ values for different cancer cell lines for Comparative Examples 1 to 3.

| Comparative | | | | | |
|---|---|---|---|---|---|
| Example | Compound | H630 WT | H630 R10 | MDA-MB-231 | A549 |
| 1 | DS (μM) | | | >8,000 | |
| 2 | (DDC)₂Cu (μM) | 5.9 ± 1.1 | 4.9 ± 1.6 | | 3.6 ± 0.7 |
| 3 | 5FU (μM) | 4.6 ± 1.6 | 462 ± 8 | | |

Comparative Examples 1 to 3 are known anti-cancer drugs or agents having anti-cancer activity. DS is disulfiram. (DDC) 2Cu is Cu(II) bis(N,N-diethyldithiocarbamate) complex. 5-FU is fluorouracil.

### REFERENCES

A number of publications are cited above in order to more fully describe and disclose the invention and the state of the art to which the invention pertains. Full citations for these references are provided below. The entirety of each of these references is incorporated herein.
Kannappan et al. “Recent advances in repurposing disulfiram and disulfiram derivatives as copper-dependent anticancer agents” Front Mol Biosci. 2021; 8:7411316 Wehbe et al. “Development and optimisation of an injectable formulation of copper diethyldithiocarbamate, an active anticancer agent” Int. J Nanomedicine. 2017; 12; 4129-4146
JP 2009-242376 A
Horita et al. “Synthesis of new sugar derivatives and evaluation of their antibacterial activities against Mycobacterium tuberculosis” Bioorg. Med. Chem. Lett; 2009; 19 (12); 6313-6316
Gopalaswamy et al. “Chemopreventive effects of dithiocarbamates on aflatoxin B1 metabolism and formation of AFB1 adducts with glutathione” Anticancer Res. 1998; 18 (3A); 1827-1832
Aft et al. “Evaluation of 2-deoxy-D-glucose as a chemotherapeutic agent: mechanism of cell death” Br J Cancer. 2002; 87, 805-812
Shakil et al. “Using chitosan or chitosan derivatives in cancer therapy” Polysaccharides. 2021; 2; 197-816
Lewis et al. “On the interaction of copper(II) with disulfiram” Chem. Commun. 2014; 50; 13334-13337
Lee et al. “Sugar-linked dithiocarbamates as modulators of metabolic and genotoxicproperties of N-nitroso compounds” J Med Chem. 1994; 37 (19); 3154-3162
Li et al. “Protection-free synthesis of glycosyl dithiocarbamates in aqueous media by using 2-chloroimidazolinium reagent” Tetrahedron Letters. 2016; 57 (31) 3529-3531
Najlah et al. “Development of injectable PEGylated liposome encapsulating disulfiram for colorectal cancer treatment” Pharmaceutics. 2019; 11 (11); 610

1. A method of treating a subject with cancer, the method comprising administering to the subject a compound, or the pharmaceutically acceptable salts or solvates thereof, wherein the compound has the general formula (I): 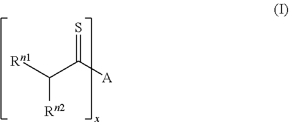
wherein each of Rⁿ¹ and Rⁿ² is independently selected from the group consisting of C₁-C₆ alkyl and C₂-C₆ alkenyl;
A is a (poly)saccharide connected via a thioglycosidic bond; and
x is 1 or more.

2. The method according to claim 1, wherein x is 1 or 2, such as x is 1.

3. The method according to claim 1, wherein each of Rⁿ¹ and Rⁿ² is independently selected from ethyl and methyl, such as each of Rⁿ¹ and Rⁿ² is ethyl or each of Rⁿ¹ and Rⁿ² is methyl.

4. The method according to claim 1, wherein A is a monosaccharide connected via a thioglycosidic bond.

5. The method according to claim 4, wherein A is 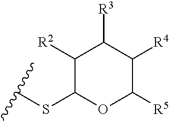
wherein each of R² to R⁴ is —OH, or wherein R² to R⁴ is each —OH and at least one —OH, such as one, is replaced with a group independently selected from H, —NH₂, acetoxy, acetylamido, —OBn and —OBz; and
R⁵ is H, or —CH₂OH, or wherein R⁵ is —CH₂OH and —OH is replaced with a group independently selected from H, —NH₂, acetoxy, acetylamido, —OBn and —OBz.

6. The method according to claim 4, wherein A is 1-thio-glycosyl connected via a thioglycosidic bond.

7. The method according to claim 4, wherein A is 1-thio-2-deoxy-glycosyl connected via a thioglycosidic bond.

8. The method according to claim 4, wherein A is 1-thio-2-N-acetylglycosylamine connected via a thioglycosidic bond.

9. The method according to claim 4, wherein A is 1-thio-3,6-diacetate-2-deoxy-glycosyl connected via a thioglycosidic bond.

10. The method according to claim 4, wherein A is 1-thio-xylosyl connected via a thioglycosidic bond.

11. The method according to claim 1, wherein A is a disaccharide, oligosaccharide or polysaccharide connected via a thioglycosidic bond.

12. The method according to claim 11, wherein A is selected from the group consisting of 4-O-galactopyranosyl-1-thio-glycosyl, 4-O-glucopyranosyl-1-thio-glycosyl, 4-O-(2-amino-2-deoxy-glucopyranosyl)-2-amino-2-deoxy-1-thio-glycosyl, 4-O-maltosyl-1-thio-glycosyl, 4-O-maltosyl-4-O-maltosyl-1-thio-glycosyl and 1-thiochitosanyl connected via a thioglycosidic bond at a terminal ring.

13. The method according to claim 11, wherein one or more-OH groups within the disaccharide, oligosaccharide or polysaccharide is each replaced with acetoxy, —OBn or —OBz.

14. A method of treating a subject with cancer, the method comprising administering to the subject a pharmaceutical composition comprising a compound of formula (I) or the pharmaceutically acceptable salts or solvates thereof according to claim 1, and a pharmaceutically acceptable carrier, or a pharmaceutically acceptable carrier selected from the group consisting of a liposome, a polymeric micelle, an emulsion, a microsphere and a nanoparticle.

15. (canceled)

16. The method according to claim 14, wherein the pharmaceutically acceptable carrier is a liposome.

17. The method according to claim 14, wherein the pharmaceutically acceptable carrier is a polymeric micelle selected from the group consisting of poly(lactic-co-glycolic acid) and polycaprolactone.

18. The method according to claim 14, wherein the pharmaceutical composition comprises a metal or a metal ion, selected from the group consisting of copper, zinc, platinum, iron, gold, silver and magnesium, and preferably copper.

19. The method according to claim 14, wherein the cancer is selected from the group consisting of colorectal cancer, breast cancer, lung cancer and brain cancer.

20. The method according to claim 19, wherein the colorectal cancer is adenocarcinoma, the breast cancer is invasive ductal carcinoma, the lung cancer is non-small cell lung cancer and the brain cancer is glioblastoma.

21. A kit comprising:
a pharmaceutical composition comprising a compound of formula (I) or the pharmaceutically acceptable salts or solvates thereof according to claim 1, and a pharmaceutically acceptable carrier, and
a metal or a metal ion, selected from the group consisting of copper, zinc, platinum, iron, gold, silver and magnesium, and preferably copper.

22.-23. (canceled)
